(19) United States
(12) Patent Application Publication
(10) Pub. No.: US 2024/0104736 A1
(43) Pub. Date: Mar. 28, 2024

(54) METHOD AND SYSTEM FOR PARALLEL PROCESSING FOR MEDICAL IMAGE

(71) Applicant: LUNIT INC., Seoul (KR)

(72) Inventor: Donggeun YOO, Seoul (KR)

(73) Assignee: LUNIT INC., Seoul (KR)

(21) Appl. No.: 18/528,923

(22) Filed: Dec. 5, 2023

Related U.S. Application Data
(63) Continuation of application No. 17/885,611, filed on Aug. 11, 2022.

(30) Foreign Application Priority Data
Aug. 11, 2021 (KR) 10-2021-0105889
Feb. 21, 2022 (KR) 10-2022-0022243

(51) Int. Cl. G06T 7/00; G06V 10/22; G06V 10/774; G16H 30/40; G16H 70/60

(52) U.S. Cl. CPC G06T 7/0012; G06V 10/22; G06V 10/774; G16H 30/40; G16H 70/60; G06T 2207/20081; G06T 2207/30004; G06V 2201/03

(57) ABSTRACT

There is provided a method for parallel processing a digitally scanned pathology image, in which the method is performed by a plurality of processors and includes performing, by a first processor, a first operation of providing a second processor with a first patch included in the digitally scanned pathology image, performing, by the first processor, a second operation of providing the second processor with a second patch included in the digitally scanned pathology image, and performing, by the second processor, a third operation of outputting a first analysis result from the first patch using a machine learning model, in which at least a part of a time frame for the second operation performed by the first processor may overlap with at least a part of a time frame for the third operation performed by the second processor.

### CROSS-REFERENCE TO RELATED APPLICATION

This application claims priority to Korean Patent Application No. 10-2021-0105889, filed in the Korean Intellectual Property Office on Aug. 11, 2021, and Korean Patent Application No. 10-2022-0022243, filed in the Korean Intellectual Property Office on Feb. 21, 2022, the entire contents of which are hereby incorporated by reference. This application is a continuation-in-part of U.S. patent application Ser. No. 17/885,611, filed on Aug. 11, 2022, the entire contents of which are hereby incorporated by reference.

### TECHNICAL FIELD

The present disclosure relates to a method and a system for parallel processing a medical image by using a plurality of processors, and more particularly, to a method and a system for outputting an analysis result from at least one patch in the medical image by using a plurality of processors.

### BACKGROUND

Recently, the development of a medical artificial intelligence solution is receiving increasing attention, which analyzes a wide range of medical data including medical image data and pathology images such as X-ray image data, computer tomography (CT) image data, and magnetic resonance imaging (MRI) data, and helps medical practitioners diagnose. Such medical artificial intelligence solution is expected to contribute to resolving the current trend in which medical staff supply becomes short while medical demand increases due to rapid aging of the population.

Meanwhile, accurate medical image reading and medical diagnosis are important to increase the utilization of the medical artificial intelligence solution, but it is also important to provide patients in the medical environment with the information quickly and rapidly. However, because medical data is generally a high-resolution image that has a very large data size, and because it requires the processing of complicated computations in the computing system, it may take a considerable amount of time until the results are derived. Accordingly, designing a fast and efficient medical image processing method can provide further advancement of the medical artificial intelligence solution.

### SUMMARY

In order to address one or more problems (e.g., the problems described above and/or other problems not explicitly described herein), the present disclosure provides a method for parallel processing a medical image by using a plurality of processors.

The present disclosure may be implemented in a variety of ways, including a method, an apparatus (or system), or a non-transitory computer-readable recording medium storing instructions.

There is provided a method for parallel processing a digitally scanned pathology image, which may be performed by a plurality of processors and include performing, by a first processor, a first operation of providing a second processor with a first patch included in the digitally scanned pathology image, performing, by the first processor, a second operation of providing the second processor with a second patch included in the digitally scanned pathology image, and performing, by the second processor, a third operation of outputting a first analysis result from the first patch using a machine learning model. At least a part of a time frame for the second operation performed by the first processor may overlap with at least a part of a time frame for the third operation performed by the second processor.

The method may further include performing, by the second processor, a fourth operation of outputting a second analysis result from the second patch using the machine learning model, in which medical information associated with the digitally scanned pathology image may be generated based on the first analysis result and the second analysis result.

The first and second patches may be included in one batch.

The performing the first operation may include extracting a first patch from a digitally scanned pathology image, and the performing the second operation may include extracting the second patch from the digitally scanned pathology image, and the first and second patches may be different from each other.

The performing the first operation may include acquiring, as the first patch, one of a plurality of patches previously extracted from the digitally scanned pathology image and stored in a storage medium, the performing the second operation includes acquiring, as the second patch, one of a plurality of patches previously extracted from the digitally scanned pathology image and stored in the storage medium, and the first and second patches may be different from each other.

There is provided a method for parallel processing a digitally scanned pathology image, in which the method may be performed by a plurality of processors and include performing, by one or more first processors, a first operation of providing one or more second processors with a first batch associated with a digitally scanned pathology image, in which the first batch may include a first set of patches, performing, by one or more third processors, a second operation of providing the one or more second processors with a second batch associated with the digitally scanned pathology image, in which the second batch may include a second set of patches, and performing, by the one or more second processors, a third operation of outputting a first analysis result from the first batch using a machine learning model. At least a part of a time frame for the second operation performed by the one or more third processors may overlap with at least a part of a time frame for the third operation performed by the one or more second processors.

The method may further include calculating a throughput required for the first operation, the second operation, and the third operation, acquiring, from a given plurality of processors, a status of operations to be processed by each of the plurality of processors at a specific point in time, and allocating one or more processors of the plurality of processors to process each of the first operation, the second operation, and the third operation, based on the calculated throughput and the acquired status of the operations. The one or more processors allocated to process the first operation may be the one or more first processors, the one or more processors allocated to process the second operation may be the one or more third processors, and the one or more processors allocated to process the third operation may be the one or more second processors.

The plurality of processors may include one or more processors in active state and one or more processors in inactive state, the acquiring the status of the operation may include calculating a maximum throughput that can be processed by the one or more processors in active state at the time of request for processing of each of the first operation, the second operation, and the third operation, and the allocating one or more processors may include determining deactivation of each of the one or more processors in active state or re-activation of each of the one or more processors in inactive state based on the calculated throughput, the calculated maximum throughput, and a target processing time.

The determining the deactivation of each of the one or more processors in active state or the re-activation of each of the one or more processors in inactive state may include if determining re-activation of each of the one or more processors in inactive state, applying a power to each of the one or more processors for which re-activation is determined, and if determining the deactivation of each of the one or more processors the previously active state, turning off the power of each of the one or more processors for which the deactivation is determined.

The target processing time may be determined differently according to a service level agreement of a user who is using the method for parallel processing digitally scanned pathology image.

The performing the first operation may include extracting the first set of patches from the digitally scanned pathology image to generate the first batch, and the performing the second operation includes extracting the second set of patches from the digitally scanned pathology image to generate the second batch. The first set of patches and the second set of patches may be different from each other.

The performing the first operation may include acquiring the first set of patches from a plurality of patches extracted from the digitally scanned pathology image, in which the extracted plurality of patches may be previously stored in a storage medium, and generating the first batch using the acquired first set of patches, and the performing the second operation may include acquiring the second set of patches from a plurality of patches extracted from the digitally scanned pathology image, in which the extracted plurality of patches may be previously stored in a storage medium, and generating the second batch using the acquired second set of patches. The first set of patches and the second set of patches may be different from each other.

At least a part of the one or more first processors may be the same as at least a part of the one or more third processors.

An information processing system may be provided, which may include a memory, and one or more first processors, one or more second processors, and one or more third processors, which may be connected to the memory and configured to execute at least one computer-readable program included in the memory, in which the one or more programs may include instructions for performing, by one or more first processors, a first operation of providing one or more second processors with a first batch associated with a digitally scanned pathology image, in which the first batch may include a first set of patches, performing, by one or more third processors, a second operation of providing the one or more second processors with a second batch associated with the digitally scanned pathology image, in which the second batch may include a second set of patches, and performing, by the one or more second processors, a third operation of outputting a first analysis result from the first batch by using a machine learning model, and at least a part of a time frame for the second operation performed by the one or more third processors may overlap with at least a part of a time frame for the third operation performed by the one or more second processors.

According to some examples, by parallel processing a medical image, the amount of operations and computations that one processor has to handle can be reduced.

According to some examples, by parallel processing a medical image, the time required for generating medical information from the medical image by using the machine learning model or training the machine learning model can be reduced.

According to some examples, by selectively extracting only the patches associated with a specific object from the medical image and using the same as training data, the performance of a machine learning model for inferring an analysis result associated with the specific object can be significantly improved.

According to some examples of the present disclosure, the time required for analyzing a medical image can be shortened by performing the operation to extract and store patches of a specific resolution in advance for the patches of different resolution levels included in the medical image.

According to some examples of the present disclosure, by processing the patches included in the medical image in parallel through asynchronous operations on a patch basis, the time required for processing the medical image can be shortened or minimized and server operating costs can be significantly reduced.

According to some examples of the present disclosure, the orchestrator can determine the status of the operations to be processed by each processor in real time and adjust the operation scale of processors to process subsequent operations, thereby maximizing the efficiency of operation processing.

According to some examples of the present disclosure, the orchestrator manages the operation scale of processors to process the medical images differently according to the service level agreements of the user, allowing users to select a method for parallel processing medical images that is suitable for them.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 illustrates an example of generating medical information associated with a medical image from the medical image;

FIG. 2 illustrates an example of an information processing system for performing parallel processing of a medical image;

FIGS. 3A, 3B, and 3C illustrate an example of parallel processing a medical image using two processors;

FIG. 4 illustrates an example of parallel processing a medical image using N number of processors;

FIG. 5 illustrates an example of generating a training batch by extracting a set of training patches from a plurality of reference medical images;

FIG. 6 illustrates an example of generating a batch by extracting a set of patches from one medical image;

FIG. 7 illustrates an example of reconstructing an image using one batch;

FIG. 8 illustrates an example of a method for parallel processing a medical image;

FIG. 9 illustrates another example of a method for parallel processing a medical image;

FIG. 10 is a structural diagram illustrating an artificial neural network model;

FIG. 11 is an exemplary system configuration for performing parallel processing of a medical image;

FIGS. 12A and 12B illustrate examples of parallel processing a medical image through asynchronous operations on a patch basis;

FIG. 13 is a structural diagram illustrating an example of an orchestrator;

FIG. 14 illustrates an example of parallel processing medical images using the orchestrator;

FIGS. 15A to 15C illustrate an example of allocating processors based on the service level agreement of the user;

FIG. 16 is a flowchart illustrating an example of a method for parallel processing medical images by asynchronous operations on a patch basis; and

FIG. 17 is a flowchart illustrating an example of a method for parallel processing medical images using an orchestrator.

### DETAILED DESCRIPTION

Hereinafter, example details for the practice of the present disclosure will be described in detail with reference to the accompanying drawings. However, in the following description, detailed descriptions of well-known functions or configurations will be omitted when it may make the subject matter of the present disclosure rather unclear.

In the accompanying drawings, the same or corresponding components are assigned the same reference numerals. In addition, in the following description of the various examples, duplicate descriptions of the same or corresponding components may be omitted. However, even if descriptions of components are omitted, it is not intended that such components are not included in any embodiment.

Advantages and features of the disclosed examples and methods of accomplishing the same will be apparent by referring to examples described below in connection with the accompanying drawings. However, the present disclosure is not limited to the examples disclosed below, and may be implemented in various forms, and the examples are merely provided to make the present disclosure complete, and to fully disclose the scope of the invention to those skilled in the art to which the present disclosure pertains.

The terms used herein will be briefly described prior to describing the disclosed embodiment(s) in detail. The terms used herein have been selected as general terms which are widely used at present in consideration of the functions of the present disclosure, and this may be altered according to the intent of an operator skilled in the art, related practice, or introduction of new technology. In addition, in specific cases, certain terms may be arbitrarily selected by the applicant, and the meaning of the terms will be described in detail in a corresponding description of the embodiment(s). Therefore, the terms used in the present disclosure should be defined based on the meaning of the terms and the overall content of the present disclosure rather than a simple name of each of the terms.

As used herein, the singular forms “a,” “an,” and “the” are intended to include the plural forms as well, unless the context clearly indicates the singular forms. Further, the plural forms are intended to include the singular forms as well, unless the context clearly indicates the plural forms. Further, throughout the description, when a portion is stated as “comprising (including)” a component, it intends to mean that the portion may additionally comprise (or include or have) another component, rather than excluding the same, unless specified to the contrary.

Further, the term “module” or “unit” used herein refers to a software or hardware component, and “module” or “unit” performs certain roles. However, the meaning of the “module” or “unit” is not limited to software or hardware. The “module” or “unit” may be configured to be in an addressable storage medium or configured to play one or more processors. Accordingly, as an example, the “module” or “unit” may include components such as software components, object-oriented software components, class components, and task components, and at least one of processes, functions, attributes, procedures, subroutines, segments of program code, drivers, firmware, micro-codes, circuits, data, database, data structures, tables, arrays, or variables. Furthermore, functions provided in the components and the “modules” or “units” may be combined into a smaller number of components and “modules” or “units”, or further divided into additional components and “modules” or “units.”

The “module” or “unit” may be implemented as a processor and a memory. The “processor” should be interpreted broadly to encompass a general-purpose processor, a central processing unit (CPU), a microprocessor, a digital signal processor (DSP), a controller, a microcontroller, a state machine, and so forth. Under some circumstances, the “processor” may refer to an application-specific integrated circuit (ASIC), a programmable logic device (PLD), a field-programmable gate array (FPGA), and so on. The “processor” may refer to a combination for processing devices, e.g., a combination of a DSP and a microprocessor, a combination of a plurality of microprocessors, a combination of one or more microprocessors in conjunction with a DSP core, or any other combination of such configurations. In addition, the “memory” should be interpreted broadly to encompass any electronic component that is capable of storing electronic information. The “memory” may refer to various types of processor-readable media such as random access memory (RAM), read-only memory (ROM), non-volatile random access memory (NVRAM), programmable read-only memory (PROM), erasable programmable read-only memory (EPROM), electrically erasable PROM (EEPROM), flash memory, magnetic or optical data storage, registers, and so on. The memory is said to be in electronic communication with a processor if the processor can read information from and/or write information to the memory. The memory integrated with the processor is in electronic communication with the processor.

In the present disclosure, an “accelerator” may refer to any processor for performing an operation required in a machine learning model, and for example, the “accelerator” may refer to a graphic processing unit (hereinafter referred to as “GPU”), a field programmable gate array (hereinafter referred to as “FPGA”), and the like, but is not limited thereto.

In the present disclosure, a “system” may refer to at least one of a server device and a cloud device, but not limited thereto. For example, the system may include one or more server devices. In another example, the system may include one or more cloud devices. In still another example, the system may include both the server device and the cloud device operated in conjunction with each other.

In the present disclosure, a “medical image” may refer to any image associated with a human body, human tissue and/or human specimen. For example, the medical image may be a 2D image or a 3D image. The medical image may include a 2D frame and/or a 3D frame.

The medical image may include a “pathology image” obtained by capturing an image of a tissue removed from the human body. In an example, the pathology image may refer to an image obtained by capturing an image of a pathology slide that is fixed and stained through a series of chemical treatments with respect to the tissue removed from the human body. In addition, the pathology image may refer to a whole slide image (WSI) including a high-resolution image with respect to an entire slide, and at least one of hematoxylin & eosin (H&E) stained slide or immunohistochemistry (IHC) stained slide. The pathology image may refer to a part of the entire high resolution slide image, such as one or more patches, for example. For example, the pathology image may refer to a digital image captured or scanned through a scanning device (e.g., a digital scanner, and the like), and may include information of specific proteins, cells, tissues, and/or structures in the human body. Further, the pathology image may include one or more patches, and the one or more patches may be applied (e.g., tagged) with the histological information through annotation work.

In another embodiment, the medical image may refer to an X-ray image, a computed tomography (CT) image, or a magnetic resonance imaging (MRI) image obtained by scanning or capturing an image of a human body.

In the present disclosure, the “patch” may refer to a small region in the pathology slide image. In an example, the patch may include a training patch used in the process of training the machine learning model and/or a patch used in the inference process of the machine learning model. For example, the patch may include a region corresponding to a semantic object extracted by performing segmentation on a medical image. As another example, the patch may refer to a combination of pixels associated with the histological information generated by analyzing the medical image.

In the present disclosure, a “batch” may include a set of patches. In an example, “a set” may refer to one or more patches.

In the present disclosure, the “machine learning model” may include any model that is used to infer an answer to a given input. In the present disclosure, various machine learning methods such as supervised learning, unsupervised learning, reinforcement learning, and the like may be used. The machine learning model may include an artificial neural network model including an input layer, a plurality of hidden layers, and an output layer. In an example, each layer may include one or more nodes. For example, the machine learning model may be trained to infer an analysis result (e.g., histological information, and the like) for a medical image and/or at least one patch included in the medical image. In this case, the histological information generated through the annotation work may be used for training the machine learning model. As another example, the machine learning model may be trained to infer an analysis result and/or medical information based on characteristics of at least one of a cell, a tissue, or a structure in the medical image. In addition, the machine learning model may include weights associated with a plurality of nodes included in the machine learning model. In an example, the weights may include any parameter associated with the machine learning model.

In the present disclosure, the machine learning model may refer to an artificial neural network model, and the artificial neural network model may refer to the machine learning model.

In the present disclosure, “training” may refer to any process of changing weights included in the machine learning model using at least one or more of medical images, patches, batches, and/or analysis results. The training may refer to a process of changing or updating weights associated with the machine learning model through one or more of forward propagation and backward propagation of the machine learning model by using at least one patch, batch and/or analysis result. In the present disclosure, “each of a plurality of A” may refer to each of all components included in the plurality of A, or may refer to each of some of the components included in a plurality of A. For example, each of a plurality of reference medical images may refer to each of all reference medical images included in a plurality of reference medical images, or to each of some reference medical images included in a plurality of reference medical images. Similarly, each of a plurality of patches may refer to each of all patches included in a plurality of patches, or may refer to each of some patches included in a plurality of patches.

In the present disclosure, the “analysis result” may include at least one of genetic information included in at least a partial region (e.g., patch, batch, and the like) of a medical image, characteristic or information of a specific protein, cell, tissue, and/or structure within the human body. The analysis result may refer to any characteristic or information of at least one patch included in the medical image, which may be inferred through the machine learning model. On the other hand, the ground truth data of the analysis result may be obtained as a result of the annotation by an annotator.

In the present disclosure, the “medical information” may refer to any medically meaningful information that can be extracted from the medical image, including, for example, regions, locations or sizes of tumor cells in the medical image, cancer diagnosis information, information associated with cancer risk of a patient, and/or medical conclusion associated with cancer treatment, or the like, but not limited thereto. In addition, the medical information may include not only quantified numerical values that can be obtained from the medical image, but also visualized information of the numerical values, prediction information according to the numerical values, image information, statistical information, or the like. The generated medical information may be provided to a user terminal, or output or transmitted to and displayed on a display device.

In the present disclosure, “spatial information of a medical image” may include information of a position corresponding to the pixel, the patch, and/or the batch in the medical image.

In the present disclosure, “statistical information of a medical image” may refer to any information that statistically indicates numerical values/distributions associated with objects (e.g., tumor cells, immune cells, and the like) included in the medical image, correlation between the objects included in the medical image, or the like.

In the present disclosure, “data” may refer to the medical image itself, information related to the medical image, the medical information, or a combination of the medical image and information.

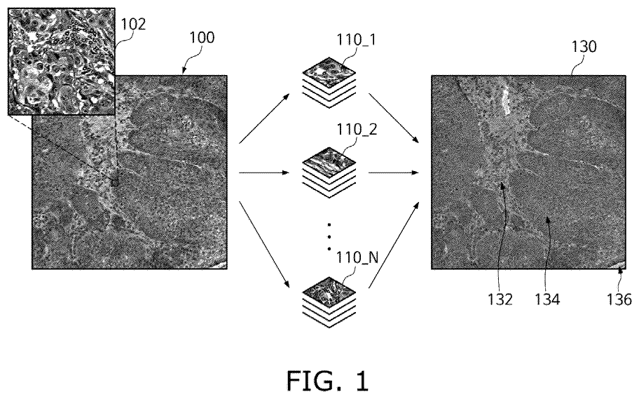

FIG. 1 illustrates an example of outputting medical information 130 associated with a medical image 100 from the medical image 100. The medical image 100 may be an image of a tissue removed from the patient's body, may be captured or scanned by using a digital scanner, and may include information of specific proteins, cells, tissues and/or structures in the patient. Further, the medical information 130 associated with the medical image 100 may refer to any information associated with a patient that can be analyzed from the medical image 100, and may include information associated with at least one of normal cells, normal epithelium, normal stroma, tumor cell (or cancer cell), cancer epithelium, cancer stroma, immune cell (lymphocyte cell), necrosis, fat, and background, although not limited thereto.

An information processing system (not illustrated) may receive the medical image 100 and extract a plurality of patches (e.g., a patch 102) from the received medical image 100. Then, the information processing system may generate a plurality of batches 110_1 to 110_N (where N is a natural number greater than 1) including a plurality of extracted patches. In this case, the patch may be set to a predetermined size (e.g., a×b pixels, where a and b are natural numbers). Further, the size of the patch may be arbitrarily changed by user input. For example, the size of the patch may be set differently according to a specific protein, cell, tissue, structure, and the like which will be included in the medical information 130 to be extracted from the medical image 100.

The information processing system may derive an analysis result from the plurality of generated batches 110_1 to 110_N, and generate or output the medical information 130 based on the analysis result. That is, a plurality of operations for the plurality of batches 110_1 to 110_N may be performed to generate the medical information 130. In an example, the operations may include all graphic processing, computing processing, training of an artificial neural network model and/or inference using the artificial neural network model, which can be performed with respect to the medical image 100 and/or the plurality of batches 110_1 to 110_N. The analysis result and/or the medical information 130 may be output or provided to the user terminal (not illustrated) and displayed on the display device associated with the user terminal (e.g., the display device connected to the user terminal, or the like). Additionally or alternatively, the information processing system may output such analysis result and/or the medical information 130 to the display device included in or connected to the information processing system. Through the display device, the analysis result and/or the medical information 130 output by the information processing system may be provided to the user.

Generally, if the information processing system performs a plurality of operations for the plurality of batches 110_1 to 110_N, the plurality of operations may be serially performed by a single processor. Therefore, it may take a relatively long time to generate the medical information 130 from the medical image 100.

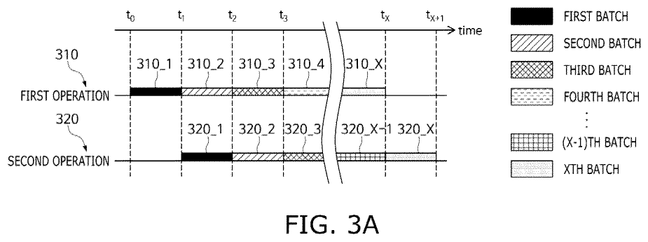

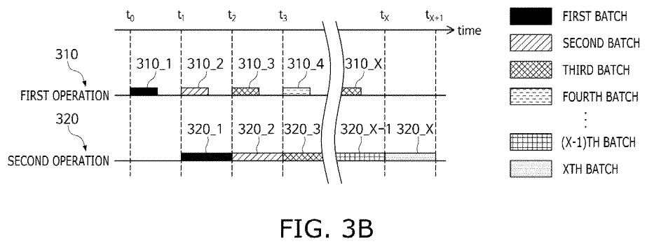

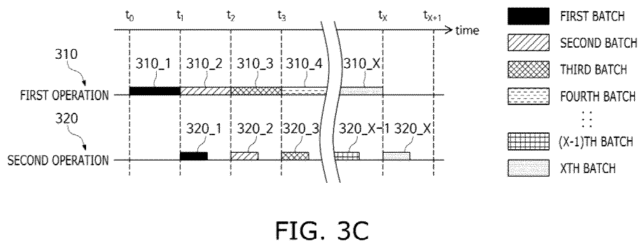

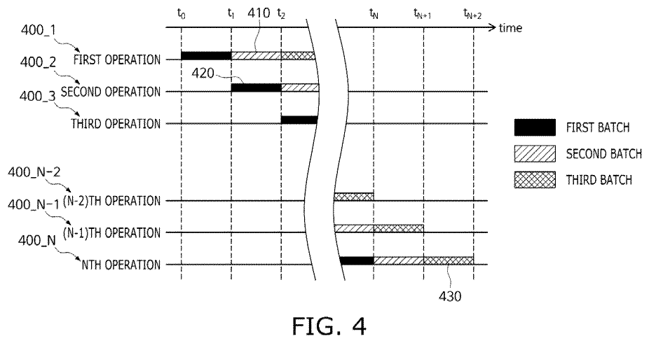

Conversely, a plurality of processors (e.g., CPU, accelerator or the like) of the information processing system may perform a plurality of operations, thereby performing the plurality of operations in parallel. Accordingly, the time required for generating the medical information 130 by the plurality of processors may be significantly shorter than the time required for generating the medical information 130 by a single processor. In addition, since a plurality of operations is distributed to each of a plurality of processors, the amount and/or time of work to be processed by one processor can also be reduced. This parallel processing will be described in detail below with reference to FIGS. 3A to 3C and 4.

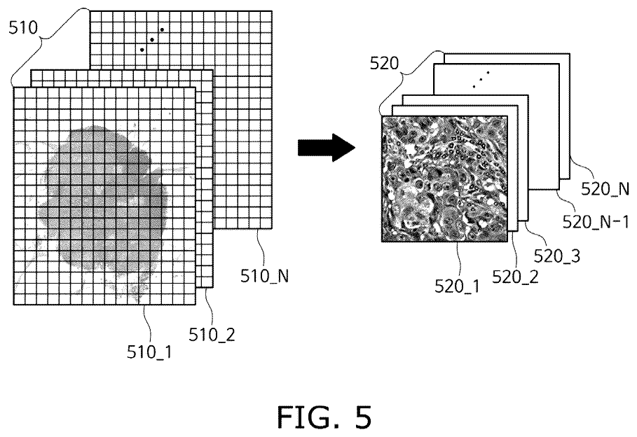

Each of a plurality of patches extracted from the medical image may include spatial information on the medical image 100. For example, the patch may include data associated with a two-dimensional coordinate value on the medical image 100. In another example, the patch may include data for a unique identification number indicating a location of the patch on the medical image 100. Such spatial information may be used in the process of generating the batches 110_1 to 110_N. In addition, the information processing system may generate or output the medical information 130 based on the spatial information and the analysis result of a plurality of batches. For example, the spatial information on the medical image 100 may be used in the process of reconstructing a plurality of patches divided from the medical image 100 to generate the medical information 130. This will be described in detail below with reference to FIGS. 5 to 7.

The medical information 130 generated from the medical image 100 may include information of characteristics or types of a tissue and/or a cell corresponding to each pixel included in the medical image 100. The medical information 130 may further include color information corresponding to the characteristics or the types of the tissue and/or the cell. For example, the medical information 130 may include a cancer stroma tissue region 132 displayed in green and a cancer epithelial tissue region 134 displayed in blue. Further, the medical information 130 may include a region 136 indicating an empty space displayed in orange. Such medical information 130 may be output or provided to the user terminal and displayed on the display device connected to the user terminal, or displayed on the display device connected to the information processing system. The medical information may include information of any tissues and/or cells of a patient distinguished by any numbers and colors corresponding thereto. Additionally or alternatively, the medical information may include information of various graphic elements, such as any shapes, any texts or the like corresponding to the classified cells and/or tissues of the patient.

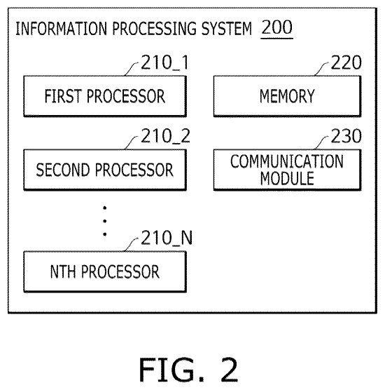

FIG. 2 illustrates an example of an information processing system 200 for performing parallel processing of a medical image. As illustrated, the information processing system 200 may include a plurality of processors 210_1, 210_2, . . . , 210_N (N is a natural number greater than 1), a memory 220 and a communication module 230. As illustrated in FIG. 2, the information processing system 200 may be configured to communicate information and/or data to an external device (e.g., the user terminal) using the communication module 230 via a network.

The memory 220 may include any non-transitory computer-readable recording medium. The memory 220 may include a permanent mass storage device such as random access memory (RAM), read only memory (ROM), disk drive, solid state drive (SSD), flash memory, and so on. In another example, a non-destructive mass storage device such as ROM, SSD, flash memory, disk drive, and so on may be included in the information processing system 200 as a separate permanent storage device that is distinct from the memory 220. Further, the memory 220 may store an operating system and at least one program code (e.g., a code for a program and/or application installed in the user terminal and providing a service related with the medical image).

These software components may be loaded from a computer-readable recording medium separate from the memory 220. Such a separate computer-readable recording medium may include a recording medium directly connectable to the information processing system 200, and may include a computer-readable recording medium such as a floppy drive, a disk, a tape, a DVD/CD-ROM drive, a memory card, and the like, for example. As another example, the software components may be loaded into the memory 220 through the communication modules rather than the computer-readable recording medium. For example, at least one program may be loaded into the memory 220 based on a computer program installed by the files provided by a developer or a file distribution system that distributes an installation file of an application via a network connected to the information processing system 200.

The plurality of processors 210_1 to 210_N may be configured to process instructions of the computer program by performing basic arithmetic, logic, and input and output operations. The plurality of processors 210_1 to 210_N may include two or more processors. The instructions may be provided to the plurality of processors 210_1 to 210_N by the memory 220 or the communication module 230. The plurality of processors 210_1 to 210_N may be configured to execute the received instructions according to a program code stored in a recording device such as the memory 220. For example, at least some of the plurality of processors 210_1 to 210_N may include an accelerator.

The communication module 230 may provide a configuration or a function for the information processing system 200 to communicate with the external device such as the user terminal and/or another system (e.g., a separate cloud system, and the like) via a network (not illustrated). For example, a request or data (e.g., a request to process medical image, a request to output medical information, and the like) generated according to a program code stored in the external device may be transmitted to the information processing system 200 via the network under the control of the communication module 230. Conversely, a control signal or instruction provided under the control of at least one of the plurality of processors 210_1 to 210_N of the information processing system 200 may be provided through the communication module 230 and network and received by the external device and/or another system via the communication module of the external device and/or another system.

Meanwhile, although not illustrated in FIG. 2, the information processing system 200 may further include an input and output interface (not illustrated). The input and output interface may be means for interfacing with an input or output device (not illustrated) that may be connected to, or included in the information processing system 200. For example, the input device may include a sensor such as a keyboard, a microphone, a mouse, an audio sensor, an image sensor, and the like, and the output device may include a device such as a display, a speaker, and the like. The input and output interface may be included in the information processing system 200 or may be configured as a separate device and connected to the information processing system 200 by wire or wirelessly. The information processing system 200 may include more components than those illustrated in FIG. 2. Meanwhile, most of the related components may not necessarily require exact illustration.

The plurality of processors 210_1 to 210_N of the information processing system 200 may be configured to manage, process, and/or store the information and/or data received from the external device and/or the external system. The information and/or data processed by the plurality of processors 210_1 to 210_N may be provided to the external device and/or the external system via a network connected to the communication module 230 and the information processing system 200. The plurality of processors 210_1 to 210_N of the information processing system 200 may receive a request for information for the processing of medical image from the external device and/or the external system via the network connected to the communication module 230 and the information processing system 200. The information processing system 200 may receive a plurality of medical images from an external memory via the network. The plurality of processors 210_1 to 210_N of the information processing system 200 may be configured to output the processed information and/or data through the output device such as a display output device (e.g., a touch screen, a display, and the like), an audio output device (e.g., a speaker), and the like, which may be included in the external device and/or the external system.

Each of the plurality of processors 210_1 to 210_N may be configured to process the operations for parallel processing of a medical image. In an example, at least some of the plurality of processors 210_1 to 210_N (i.e., the first processor) may perform the operation of extracting a set of patches from the image to generate a batch and providing the generated batch to a second processor. In an example, the first processor may refer to CPU, and the second processor may refer to one or more of the plurality of processors 210_1 to 210_N that do not process the operation described above. For example, it may refer to an accelerator for processing computation through the machine learning model. Specifically, the second processor may perform the operation of outputting an analysis result from the received batch using the machine learning model. The operation performed as described above may be stored in a storage medium or provided to another processor (e.g., the first processor or another processor) for generating medical information associated with the medical image based on analysis result.

The number of patches included in the batch generated by the first processor may be two or more. Using a batch including two or more patches for the training and/or inferring process of the machine learning model can provide much faster speed than using one patch. For example, if it is assumed that there are 1000 patches, inputting a batch including 100 patches into the machine learning model for ten times is faster than inputting these patches into the machine learning model one by one for 1000 times in terms of the total processing time taken to generate the analysis results for the batch by using an accelerator.

According to another example, when the machine learning model is implemented as a deep neural network, the statistical amount of a plurality of patches included in the batch may be used to train the machine learning model. For example, in the Batch Normalization layer, the statistical information such as an average vector and a variance vector may be extracted using a feature map in the intermediate stage, which is generated by inputting the batches included in the batch to the deep neural network. Such information can be used in the training of the machine learning model, thus greatly improving the performance of the machine learning model.

FIGS. 3A to 3C illustrate an example of parallel processing a medical image using two processors. As illustrated, X number of batches (X is a natural number of 2 or more) may be extracted from the medical image. Each batch may include a plurality of patches. In this case, X number of batches may be the batches extracted from one medical image, or the batches extracted from a plurality of medical images. In an example, the medical image may be stored in a memory accessible by the first processor (e.g., an internal memory of the information processing system or a memory included in the external device). While FIGS. 3A to 3C illustrate that six or more batches are extracted, aspects are not limited thereto. For example, one to five batches may be extracted from the medical image.

A first operation 310 and a second operation 320 may be performed by different processors. For example, the first operation 310 may be performed by the first processor (e.g., CPU, not illustrated), and the second operation 320 may be performed by the second processor (e.g., accelerator, not illustrated). Additionally, the first operation 310 and the second operation 320 may refer to different operations for the medical image. For example, the first operation 310 may include extracting a batch including a set of patches from the medical image and providing the extracted batch to the second processor. Additionally, the first operation 310 may include a preprocessing operation of the medical image. In an example, the pre-processing operation of the medical image may refer to any pre-processing operation that enhances quality of the medical image for the purpose of processing and analysis of the medical image, but aspects are not limited hereto, and it may refer to at least one of contrast adjustment, brightness adjustment, saturation, blur adjustment, noise injection, random crop, and sharpening.

The second operation 320 may perform an operation of outputting an analysis result for a corresponding batch from the received batch including a set of patches, by using the machine learning model. The second operation 320 may refer to any computation associated with the machine learning model for outputting the analysis result by using the received batch as input data.

If the machine learning model is trained by the second processor, the second operation 320 may include an operation of training the machine learning model by the second processor. To this end, the second processor may input each of a plurality of batches in a plurality of medical pathology images into the machine learning model, and receive an analysis result for each of a plurality of batches, for example. In this case, the second processor may calculate a loss by comparing the analysis result for each of a plurality of batches with the ground truth result (i.e., a plurality of reference analysis results) of the batch corresponding to each of a plurality of batches, and perform back-propagation of the calculated loss with the machine learning model so as to train the machine learning model. According to another example, if the second processor performs a validation operation of the machine learning model or a prediction operation through the machine learning, the second operation 320 may include an operation of outputting an analysis result corresponding to each of one or more batches through the machine learning model. In addition, the second operation 320 may include an operation of generating medical information based on the output analysis result. The result of the second operation processed as described above may be provided to the storage medium and/or the first processor.

The first processor may collect the analysis results for a plurality of batches, and generate medical information associated with the medical image based on the collected analysis results. Alternatively, at least a part of the operation of generating such medical information may be performed by the second processor or by a processor other than the first processor and the second processor. For example, it is possible to prevent one processor from computational overloads by having the first processor perform the operation of collecting the analysis result and having the second processor perform the operation of performing additional computation from the analysis result.

In an example, each of a plurality of patches included in or associated with a plurality of batches may include spatial information on the medical image (e.g., information indicating position in the medical image). The first processor may generate a processed image based on the spatial information of each of a plurality of patches. In an example, the processed image may refer to an image reconstructed by dividing the received medical images in the units of batches and then combining the divided batches. Medical information associated with the medical image may be generated based on the first analysis result, the second analysis result, and the processed image. In an example, the generated medical information may be included in the processed image. That is, the processed or reconstructed image may be an image restored by combining the analysis results corresponding to the patches or batches.

FIG. 3A is a first example of parallel processing a medical image. From t₀ to t₁, the first processor may perform a first operation 310_1 for a first batch, by extracting a first set of patches from one or more medical images to generate the first batch, and providing the generated first batch to the second processor. From t₁ to t₂, the first processor may perform a first operation 310_2 for a second batch, by extracting a second set of patches from one or more medical images to generate the second batch, and providing the generated second batch to the second processor. Further, from t₁ to t₂, the second processor may perform a second operation 320_1 for the first batch, by outputting a first analysis result using the first batch received from the first processor. That is, from t₁ to t₂, while the first processor is performing the first operation 310_2 for the second batch, the second processor may perform the second operation 320_1 for the first batch. The second operation may include an operation of storing the output analysis result in the storage medium.

Accordingly, the time frame for the first operation 310_2 for the second batch may overlap with the time frame for the second operation 320_1 for the first batch performed by the second processor. That is, while the first processor is performing the first operation 310_2 for the second batch, the second processor may process the second operation 320_1 for the first batch in parallel. While the first processor is performing the first operation 310_2 for the second batch, at least a part of the second operation 320_1 for the first batch of the second processor may be performed. Likewise, the time frame for the first operation 310_3 for a third batch may overlap with the time frame for the second operation 320_2 for the second batch performed by the second processor, and the time frame for the first operation 310_4 for the fourth batch may overlap with the time frame for the second operation 320_3 for the third batch performed by the second processor. That is, from tₓ₋₁ to tₓ, the first processor may perform the first operation 310_X for an Xth batch, by extracting an Xth set of patches from the one or more medical images to generate the Xth batch, and providing the generated Xth batch to the second processor. Further, from tₓ₋₁ to tₓ, the second processor may perform the second operation 320_X-1 for an (X-1)th batch, by outputting an (X-1)th analysis result by using the (X-1)th batch received from the first processor. That is, from tₓ₋₁ to tₓ, while the first processor is performing the first operation 310_X for the Xth batch, the second processor may perform the second operation 320_X-1 for the (X-1)th batch in parallel. Accordingly, the time frame for the first operation 310_X of the Xth batch may overlap with the time frame for the second operation 320_X-1 for the (X-1)th batch performed by the second processor.

From tₓ to tₓ₊₁, the first processor may not perform any operation. Further, from tₓ to tₓ₊₁, the second processor may perform the second operation 320_X for the Xth batch, by outputting the Xth analysis result by using the Xth batch received from the first processor. Additionally, after tₓ₊₁, the first processor and/or another processor (e.g., the second processor, or a processor other than the first processor and the second processor) may further perform the operation of generating medical information by using the first to Xth analysis results extracted between t₁ and tₓ₊₁. In an example, the medical information may include not only quantified numerical values that can be obtained from the medical image, but also visualized information of the numerical values, prediction information according to the numerical values, image information, statistical information, or the like. For example, the first processor and/or another processor may generate such medical information by using the machine learning model that is trained to infer the medical information based on one or more analysis results. As another example, the second processor may be configured to generate the medical information by using the first to Xth analysis results.

According to another example, the first processor and/or another processor may start the operation of receiving the analysis result for some of all batches from the second processor, and using the received analysis results for some batches to generate the medical information associated with the medical image. Such operation may be finished after the analysis results for the remaining batches are received and the received analysis results for the remaining batches are reflected in the medical information.

FIG. 3B is a second example of parallel processing a medical image. As illustrated, the time taken for the first operation 310 for one batch may be shorter than the time taken for the second operation 320 for one batch. In FIG. 3B, the configuration already described above or overlapping with FIG. 3A will not be described.

From tₓ₋₁ to tₓ, the first processor may perform a first operation 310_X for an Xth batch, by extracting an Xth set of patches from one or more medical images to generate the Xth batch, and providing the generated Xth batch to the second processor. In this case, the first operation 310_X for the Xth batch is illustrated as starting at tₓ₋₁ and ending before tₓ, but aspects are not limited thereto. For example, the first operation 310_X for the Xth batch may be performed at any time between tₓ₋₁ and tₓ. Meanwhile, from tₓ₋₁ to tₓ, the second processor may perform a second operation 320_X-1 for an (X-1)th batch, by outputting an (X-1)th analysis result by using the (X-1)th batch received from the first processor. That is, from tₓ₋₁ to tₓ, while the first processor is performing the first operation 310_X for the Xth batch, the second processor may perform the second operation 320_X-1 for the (X-1)th batch in parallel. Accordingly, the time frame for the first operation 310_X of the Xth batch performed by the first processor may overlap with at least a part of the time frame for the second operation 320_X-1 of the (X-1)th batch performed by the second processor.

FIG. 3C is a third example of parallel processing a medical image. As illustrated, the time taken for the first operation 310 for one batch may be longer than the time taken for the second operation 320 for one batch. In FIG. 3C, the configuration already described above or overlapping with FIG. 3A will not be described.

From tₓ₋₁ to tₓ, the first processor may perform the first operation 310_X for an Xth batch, by extracting an Xth set of patches from one or more medical images to generate the Xth batch, and providing the generated Xth batch to the second processor. Meanwhile, from tₓ₋₁ to tₓ, the second processor may perform the second operation 320_X-1 for an (X-1)th batch, by outputting an (X-1)th analysis result by using the (X-1)th batch received from the first processor. In an example, while the second operation 320_X-1 for the (X-1)th batch is illustrated as starting at tₓ₋₁ and ending before tₓ, aspects are not limited thereto. For example, the second operation 320_X-1 for the (X-1)th batch may be performed at any time between tₓ₋₁ and tₓ. That is, from tₓ₋₁ to tₓ, while the first processor is performing the first operation 310_X for the Xth batch, the second processor may perform the second operation 320_X-1 for the (X-1)th batch. Accordingly, at least a part of the time frame for the second operation 320_X-1 for the (X-1)th batch performed by the second processor may overlap with the time frame for the first operation 310_X for the Xth batch performed by the first processor.

FIG. 4 illustrates an example of parallel processing a medical image using N number of processors. In an example, the method for processing a medical image may be divided into N number of operations. For example, if the method for processing a medical image includes a plurality of processing steps, the N number of operations may include successive processing steps in groups. For example, a first operation 400_1 may include first and second steps, a second operation 400_2 may include a third step, a third operation 400_3 may include a fourth step, an (N-2)th operation 400_N-2 may include fifth and sixth steps, an (N-1)th operation 400_N-1 may include seventh and eighth steps, and N operations may refer to ninth to Jth steps (where, J is a natural number greater than 9). That is, any one operation may include one or more continuous processing steps.

As illustrated, the method for extracting one batch and processing the extracted batch may be divided into N number of operations. For example, the operation of extracting one set of patches from one or more medical images to generate a batch and providing the generated batch to the second processor may be included in the first operation performed by the first processor. The operation of deriving an analysis result from the batch generated by using the machine learning may be divided into (N-1) number of operations performed by (N-1) number of processors. Meanwhile, for convenience of explanation, FIG. 4 illustrates an example in which three batches are extracted from the medical image and processed, but aspects are not limited thereto. For example, one, two, or four or more batches may be extracted from the medical image. Unlike FIG. 4, the first operation of extracting one set of patches from one or more medical images to generate a batch and providing the generated batch to the second processor may be divided into several sub-operations and assigned to several processors.

As illustrated, from t₁ to t₂, the first processor may perform a first operation 410 of generating a second batch. Further, from t₁ to t₂, the second processor may perform a second operation 420 which is a part of the operation of outputting the first analysis result by using the first batch received from the first processor. That is, from t₁ to t₂, while the first processor is performing the first operation 410 for the second batch, the second processor may perform the second operation 420 for the first batch. Accordingly, at least a part of the time frame for the first operation 410 for the second batch may overlap with at least a part of the time frame for the second operation 420 for the first batch performed by the second processor. Additionally, the remaining operation of outputting the first analysis result by using the first batch may be performed by a plurality of processors (N-2 number of processors) different from the first processor and the second processor. According to such process, from t_{N+1} to t_{N+2}, the Nth processor may perform an Nth operation 430 for a third batch. In the present disclosure, it is assumed that the number of processors, that is, N is 6 or more for convenience of illustration, but aspects are not limited, and N may be any natural number equal to or less than 5.

According to another example, one processor may be configured to perform the extracting and the analyzing operations for one batch. For example, a batch at a specific time point may be referred to as Y (where, Y is a natural number), and another batch at the next time point may be referred to as (Y+1). If an Nth operation for a Y batch is being performed by the Nth processor (where, N is the number of processors involved in the processing) at a specific time point, the (N-1)th processor may perform an (N-1)th operation for a (Y+1) batch at the same time. Further, at the same time point, the (N-2)th processor may perform an (N-2)th operation for a (Y+2) batch. Likewise, at the same time point, the first processor may simultaneously perform the first operation for the (Y+N-1) patch. In the present disclosure, although it is explained that one processor performs the extracting and the analyzing operations for one batch, aspects are not limited thereto, and two or more processors may be configured to perform the extracting and the analyzing operations for one batch.

According to another example, the operation of extracting and processing each of a plurality of batches from a medical image may be divided into N number of operations. Each of a plurality of operations 400_1 to 400_N may include at least some of operations of receiving a medical image from the memory (e.g., memory 220), extracting a patch (or a batch) from the medical image, pre-processing (e.g., contrast adjustment, brightness adjustment, saturation adjustment, blur adjustment, noise injection, random crop and/or sharpening, and the like), designing and training a machine learning model and/or inferring with the machine learning model, by using a data set including the extracted patch (or batch), reconstructing the patch (or batch), or generating medical information associated with the medical image based on the analysis results. That is, at least some of a series of operations for analyzing the medical image may be grouped and assigned as one operation. The one operation assigned as described above may be assigned to one of a plurality of processors and performed at a specific time point, and at such specific time point, parallel processing may be performed, that is, another operation may be performed in another processor.

As described above, the information processing system according to various examples uses a plurality of processors to extract a plurality of patches from at least one medical image and train an artificial neural network model based on the extracted patches, or extract a plurality of patches from at least one medical image and infer the medical information from the artificial neural network model by using the extracted patches as inputs.

FIG. 5 is a view for explaining a method in which the information processing system extracts a plurality of patches from at least one medical image by using a plurality of processors and trains an artificial neural network model based on the extracted patches.

FIG. 5 illustrates an example of extracting a set of training patches 520_1 to 520_N (N is a natural number equal to or greater than 2) from a plurality of reference medical images 510 to generate a training batch 520. At least one of first to Mth processors 210_1 to 210_M (where, M is a natural number equal to or greater than 2) may train the machine learning model so as to infer the reference analysis result by using the training batch 520. Meanwhile, in the above disclosure, the terms are unified as “medical image”, “patch” and “batch”, but in FIGS. 5 to 8, they are further separated and used. Specifically, “reference medical image”, “training patch” and “training batch” may refer to data used for the training of the machine learning model, and “medical image”, “patch” and “batch” may refer to data used for the inference of the machine learning model. However, the aspects are not limited thereto, and each of “reference medical image”, “training patch”, and “training batch” may be replaced and construed as “medical image”, “patch” and “batch”. In addition, each of “medical image”, “patch”, and “batch” may be replaced and construed as “reference medical image”, “training patch”, and “training batch”.

The processor may perform segmentation for a specific object in each of the plurality of reference medical images 510. For example, the processor may perform segmentation for at least one of the objects of a tumor cell (or cancer cell), a cancer epithelial, a cancer stroma, and a lymphocyte cell. The processor may extract the set of training patches 520_1 to 520_N including the corresponding specific object to generate the training batch 520. Additionally or alternatively, the processor may extract the set of training patches 520_1 to 520_N that do not include the specific object to generate a training batch (not illustrated). With such configuration, the processor may improve performance of the machine learning model to infer an analysis result associated with the specific object. Additionally or alternatively, the processor may receive a reference medical image having segmentation information for a specific object in the reference medical image, and extract training patches including the specific object by using the reference medical image, to thus generate a training batch.

The processor may generate a training batch by extracting the patches in each of the first to Nth reference medical images 510_1 to 510_N based on the spatial information of the first to Nth reference medical images 510_1 to 510_N. From each of the first to Nth reference medical images 510_1 to 510_N, a patch located at a first point (e.g., an area including one or more pixels) may be extracted to generate the training batch 520. For example, the first training patch 520_1 may be extracted from a first point of the first reference medical image 510_1, and the second training patch 520_2 may be extracted from a first point of the second reference medical image 510_2 corresponding to the first point of the first reference medical image 510_1, and the Nth training patch 520_N may be extracted from a first point of the Nth reference medical image 510_N corresponding to the first point of the first reference medical image 510_1. Additionally, the processor may further generate a training batch (not illustrated) by extracting a patch located at a second point from each of the first to Nth reference medical images 510_1 to 510_N based on the spatial information of the first to Nth reference medical images 510_1 to 510_N. In this case, the second point may be determined based on the first point. For example, the second point may be determined to be the same as, or adjacent to the first point. In another example, the second point may be determined to be a point separated by a predetermined distance in a predetermined direction from the first point. With this configuration, the processor can simplify the calculation for reconstructing the extracted patches to generate medical information.

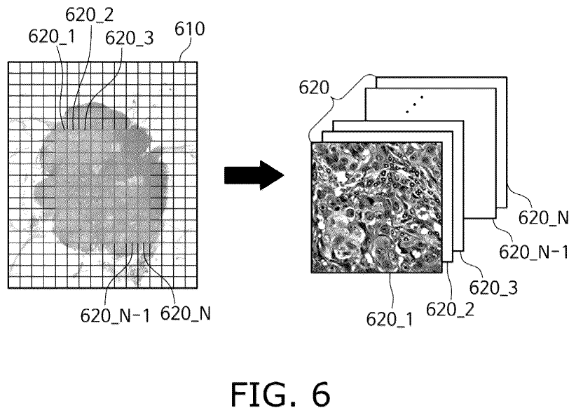

FIG. 6 is a view illustrating a method in which the information processing system extracts a plurality of patches from at least one medical image by using a plurality of processors and receives medical information from an artificial neural network model by inputting the extracted patches.

FIG. 6 illustrates an example of generating a batch 620 by extracting a set of patches 620_1 to 620_N (where, N is a natural number equal to or greater than 2) from one medical image 610. The processor may extract the first patch 620_1 from the medical image 610 to generate the batch 620. Then, the processor may extract a second patch 620_2 from the medical image 610 based on the spatial information on the medical image 610 of the first patch 620_1. Likewise, the processor may extract a third patch 620_3 from the medical image 610 based on the spatial information on the medical image 610 of the second patch 620_2. Likewise, the processor may extract the Nth patch 620_N from the medical image 610 based on the spatial information on the medical image 610 of the (N-1)th patch 620_N-1. For example, the (N-1)th patch 620_N-1 and the Nth patch 620_N may be adjacent to each other in the medical image 610. Additionally, at least a part of the (N-1)th patch 620_N-1 and at least a part of the Nth patch 620_N may overlap with each other in the medical image 610. As illustrated in FIG. 6, the batch 620 may include adjacent patches in the medical image 610, but aspects are not limited thereto. The batch 620 may include a plurality of patches which are spatially randomly extracted from the medical image 610. The generated batch 620 may be used as input data in the process of inference using the machine learning model.

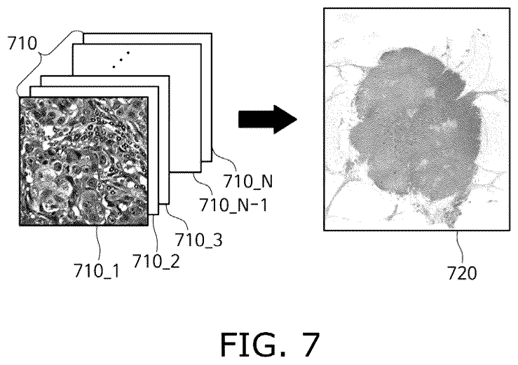

FIG. 7 illustrates an example of reconstructing a processed image 720 by using one batch 710. As described above, an analysis result may be output from a set of patches 710_1 to 710_N (where N is a natural number equal to or greater than 2) extracted from one or more medical images. The processor may reconstruct the image 720 based on the analysis result extracted from the batch 710. Accordingly, reconstructing the image 720 based on the analysis result may refer to generating a processed image by recombining the set of patches 710_1 to 710_N. Also, reconstructing the image 720 based on the analysis result may refer to generating the medical information based on the processed image and the analysis result. For example, the medical information may be provided to a user in a visualized form.

The set of patches 710_1 to 710_N included in the batch 710 may include spatial information on the medical image. For example, each of a plurality of patches included in the set of patches 710_1 to 710_N may include a two-dimensional coordinate value on the medical image. The processor may reconstruct the image 720 based on the spatial information of the set of patches 710_1 to 710_N. The reconstructed image 720 may include the analysis result and/or the medical information generated based on the analysis result.

Meanwhile, although FIG. 7 illustrates that one image 720 is reconstructed from one batch 710 for convenience of description, aspects are not limited thereto. For example, the processor may reconstruct one medical image from a plurality of batches. As another example, the processor may reconstruct a plurality of medical images from a plurality of batches. In this case, each of a plurality of patches included in the batch may include spatial information including information of the medical image to which each patch belongs, and a two-dimensional coordinate value in the corresponding medical image.

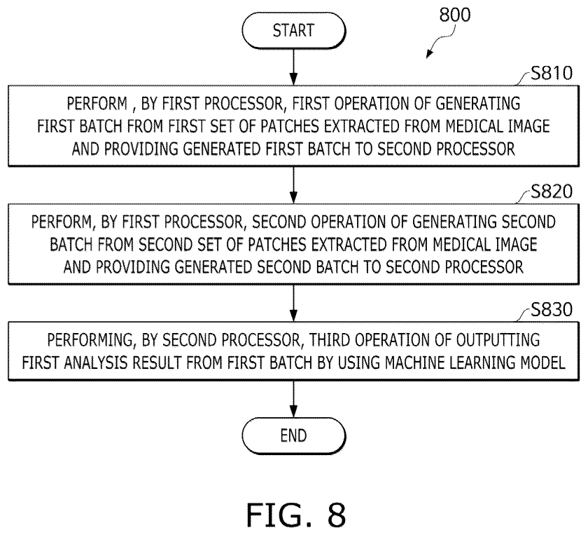

FIG. 8 illustrates an example of a method 800 for processing a medical image in parallel. The method 800 may be performed by at least some of the plurality of processors (e.g., the plurality of processors 210_1 to 210_N) illustrated in FIG. 2 of the information processing system.

The first processor of the information processing system may perform a first operation of generating a first batch from a first set of patches extracted from the medical image and providing the generated first batch to the second processor (S810). The medical image may be received from an internal or external memory of the information processing system. For example, the medical image may include a digitally scanned pathology image, an X-ray image, a CT image, or an MRI image. The first processor may perform a second operation of generating a second batch from a second set of patches extracted from the medical image (S820). In an example, each of the first set of patches and the second set of patches may include one or more patches. Further, each of the first batch and the second batch may include a first training batch and a second training batch used in a training process of the machine learning model and extracted from one or more reference medical images. In an example, the reference medical image may include a digitally scanned reference pathology image.

The medical image may include a plurality of reference medical images including a first reference medical image and a second reference medical image. The first processor may generate one batch by using the patches extracted from a plurality of reference medical images. For example, the first processor may extract x number of patches from the first reference medical image, extract y number of patches from the second reference medical image, extract z number of patches from the third reference medical image, and generate one batch including (x+y+z) number of patches. In an example, each of x, y, and z may be any of natural numbers.

The first processor may receive the digitally scanned pathology image including segmentation information of a specific object in the digitally scanned pathology image. In this case, the first processor may extract a first set of patches including the specific object and generate a first batch including the extracted first set of patches. The first processor may extract a second set of patches that do not include the specific object, and generate a second batch including the extracted second set of patches. According to another example, the first processor may extract a second set of patches including the specific object, and generate a second batch including the extracted second set of patches.

If the specific object is a cancer cell or cancer tissue, in the training process of the machine learning model, the first processor may extract the training patches so that the number of patches included in the first set of training patches including the cancer cell or cancer tissue, and the number of patches included in the second set of training patches that do not include the cancer cell or cancer tissue correspond to a predetermined number. For example, the number of training data may be properly adjusted, as the first processor extracts 50 patches for the first set of training patches including the cancer cell or cancer tissue, and extracts 50 patches for the second set of training patches that do not include the cancer cell or cancer tissue. As another example, if an area including cancer cell or cancer tissue in one or more reference medical images is insufficient to be used as the training data, the first processor may configure a batch by oversampling the patch including the cancer cell or cancer tissue. As another example, if a patch of a normal cell or tissue is extracted as the training patch to be included in the first batch and/or the second batch, the first processor may extract patches of a cancer cell or cancer tissue as the subsequent training patch. According to the method described above, by properly distributing the training data including a specific object and the training data that does not include the specific object, the machine learning model can be effectively trained.

The first set of patches belonging to the first batch and the second set of patches belonging to the second batch may be spatially associated with each other in the medical image. For example, each of at least one patch included in the first set of patches and each of at least one patch included in the second set of patches may be adjacent to each other or overlap with each other at least in part in the medical image. As another example, the first processor may extract the patches spaced apart at intervals in the medical image as the first set of patches and the second set of patches. In another embodiment, the first processor may randomly extract the first set of patches and the second set of patches from the medical image.

The second processor of the information processing system may perform a third operation of outputting the first analysis result from the first batch by using the machine learning model (S830). In this case, at least a part of the time frame for the second operation performed by the first processor may overlap with at least a part of the time frame for the third operation performed by the second processor.

The second processor may perform a fourth operation of outputting the second analysis result from the second batch by using the machine learning model. The first processor and/or another processor (i.e., a processor other than the first processor and the second processor) may generate medical information associated with the medical image based on the first analysis result and the second analysis result. While it is described herein that the medical information is generated based on the analysis results of two batches, aspects are not limited thereto, and the medical information may be generated based on the analysis results of three or more batches. In an example, the medical image may include a plurality of reference medical images used for the training process of the machine learning model.

The machine learning model may be trained by using a plurality of batches and a plurality of reference analysis results in the plurality of reference medical pathology images. The second processor may input each of a plurality of batches in a plurality of reference medical pathology images into the machine learning model, and receive an analysis result for each of a plurality of batches. In this case, the second processor may calculate a loss by comparing the analysis result for each of a plurality of batches with the ground truth result (i.e., a plurality of reference analysis results) of the batch corresponding to each of a plurality of batches, and perform back-propagation of the calculated loss with the machine learning model so as to train the machine learning model. Additionally or alternatively, such training process may be performed by the first processor. As described above, a plurality of reference medical pathology images may be used for the training process of the machine learning model, but the machine learning model may be trained by using one reference medical pathology image.

Each of a plurality of patches associated with the first batch and the second patch may include spatial information on the received reference medical image. A processed image may be generated based on the spatial information of each of a plurality of patches. Generating the processed image may be performed by the first processor and/or another processor. Medical information associated with the medical image may be generated based on the first analysis result, the second analysis result, and the processed medical image. Generating such medical information may be performed by the first processor and/or another processor. In an example, the medical information may include statistical information of the medical image.

The first processor may perform image processing on the generated first batch, and perform image processing on the generated second batch. In this case, the image processing on the first batch and the image processing on the second batch may include at least one of contrast adjustment, brightness adjustment, saturation adjustment, blur adjustment, noise injection, random cropping, or sharpening.

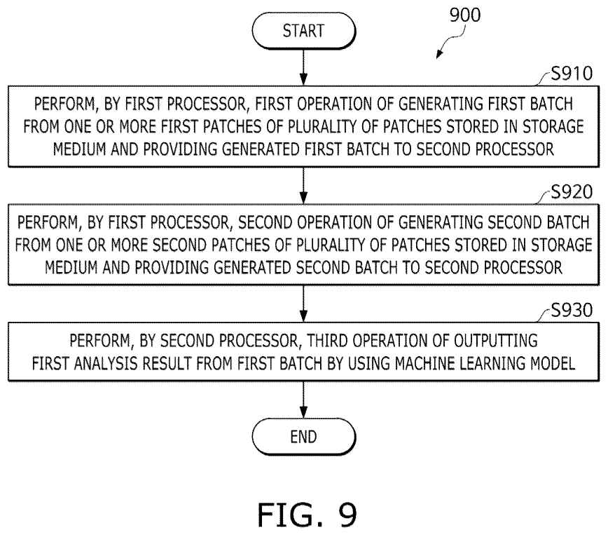

FIG. 9 illustrates another example of a method 900 for parallel processing a medical image. The method 900 may be performed by at least some of the plurality of processors (e.g., the plurality of processors 210_1 to 210_N) illustrated in FIG. 2. In FIG. 9, the configuration already described above or overlapping with FIG. 8 will not be described.

The first processor may perform a first operation of generating a first batch from one or more first patches of a plurality of patches stored in the storage medium and providing the generated first batch to the second processor (S910). Further, the first processor may perform a second operation of generating a second batch from one or more second patches of a plurality of patches stored in the storage medium and providing the generated second batch to the second processor (S920). In an example, a plurality of patches may be extracted in advance and stored in a storage medium independently of inference or training step of the machine learning model.

A plurality of patches stored in the storage medium may be extracted from the medical image. In an example, a subject that extracts a plurality of patches from the medical image may be the first processor, the second processor, another processor (a processor other than the first processor and the second processor in the information processing system), a processor included in a device separate from the information processing system and/or any combination of such processors. Further, the storage medium may be any storage medium included in the information processing system. Additionally or alternatively, the storage medium may be any storage medium that is accessible by, or coupled to the information processing system. For example, the storage medium may be a mass storage of a server device connected to the information processing system via the network, a cloud system, and/or the external device.

Further, the second processor may perform a third operation of outputting the first analysis result from the first batch by using the machine learning model (S930). In this case, at least a part of the time frame for the second operation performed by the first processor may overlap with at least a part of the time frame for the third operation performed by the second processor.

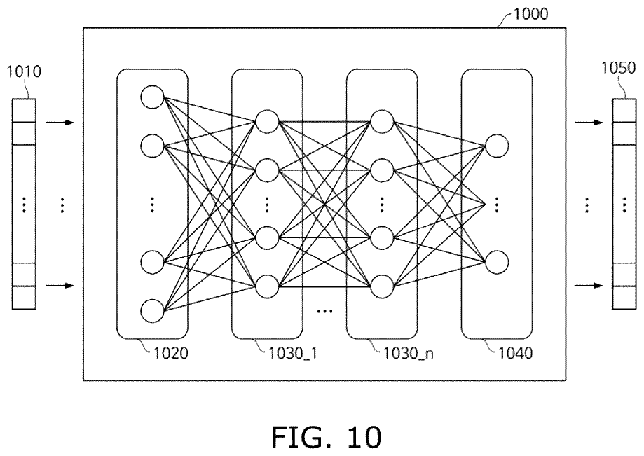

FIG. 10 is a structural diagram illustrating an artificial neural network model 1000. The machine learning model described above may refer to the artificial neural network model 1000. In the machine learning technology and cognitive science, the artificial neural network model 1000 refers to a statistical training algorithm implemented based on a structure of a biological neural network, or to a structure that executes such algorithm.

The artificial neural network model 1000 may represent a machine learning model that acquires a problem solving ability by repeatedly adjusting the weights of synapses by the nodes that are artificial neurons forming the network through synaptic combinations as in the biological neural networks, thus training to reduce errors between a target output corresponding to a specific input and a deduced output. For example, an artificial neural network model 700 may include any probability model, neural network model, and the like, that is used in artificial intelligence learning methods such as machine learning and deep learning.

The artificial neural network model 1000 may be implemented as a multi-layer perceptron (MLP) formed of multi-layer nodes and connections between them. The artificial neural network model 1000 may be implemented using one of various artificial neural network structures including the MLP. As illustrated in FIG. 10, the artificial neural network model 1000 may include an input layer 1020 receiving an input signal or data 1010 from the outside, an output layer 1040 outputting an output signal or data 1050 corresponding to the input data, and n number of hidden layers 1030_1 to 1030_n positioned between the input layer 1020 and the output layer 1040 to receive a signal from the input layer 1020, extract the characteristics, and transmit the characteristics to the output layer 1040. In an example, the output layer 1040 may receive signals from the hidden layers 1030_1 to 1030_n and output them to the outside.

The method of training the artificial neural network model 1000 includes the supervised learning that trains to optimize for solving a problem with inputs of teacher signals (correct answers), and the unsupervised learning that does not require a teacher signal. The method for parallel processing a medical image may use supervised learning, unsupervised learning, and/or semi-supervised learning to train the artificial neural network model 1000 configured to output the analysis result from one or more batches in the medical image. According to another example, the artificial neural network model 1000 for outputting medical information associated with the medical image may be trained based on a plurality of analysis results output from a plurality of batches in the medical image. The artificial neural network model 1000 trained in this way may output an analysis result and/or medical information of a patient related to a corresponding medical image.

As illustrated in FIG. 10, an input variable of the artificial neural network model 1000 that is capable of outputting an analysis result may be a vector 1010 indicating a batch including one set of patches extracted from the medical image, which includes one vector data element of the medical image. Under this configuration, the output variable may include a result vector 1050 indicating the analysis result for the input batch.

According to another example, the artificial neural network model 1000 may be trained to generate medical information according to the input variable. For example, the input variable may be the vector 1010 including vector data elements representing a plurality of analysis results for a plurality of batches included in the medical image. Under this configuration, the output variable may be configured as the result vector 1050 indicating the medical information associated with the medical image.

As described above, by matching the input layer 1020 and the output layer 1040 of the artificial neural network model 1000 with a plurality of input variables and a plurality of corresponding output variables, respectively, and adjusting synaptic values between nodes included in the input layer 1020, the hidden layer 1030_1 . . . 1030_n (where n is a natural number equal to or greater than 2), and the output layer 1040, the artificial neural network model 1000 may be trained to infer the correct output corresponding to a specific input. For the inference of a correct output, the correct answer data of the analysis result may be used, and such correct answer data may be obtained as a result of annotation by an annotator. Through this training process, the features hidden in the input variables of the artificial neural network model 1000 can be confirmed, and the synaptic values (or weights) between the nodes of the artificial neural network model 1000 can be adjusted so that there can be a reduced error between the target output and the output variable calculated based on the input variable.

Therefore, using the artificial neural network model 1000 trained as described above, the analysis result and/or medical information necessary for the medical diagnosis can be extracted from the medical image of a patient. For example, by using the artificial neural network model 1000, data and/or information associated with at least one of normal cells, normal epithelial, normal stroma, tumor cells (or cancer cell), cancer epithelial, cancer stroma, lymphocyte cell, necrosis, fat, and background can be extracted from the medical image.

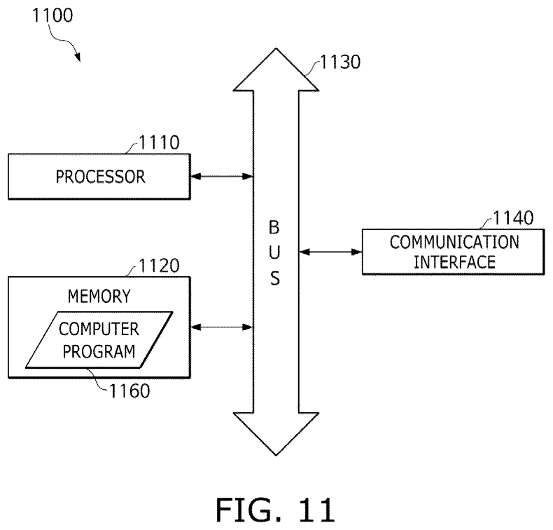

FIG. 11 is an exemplary system configuration for performing parallel processing of a medical image. An information processing system 1100 of FIG. 11 may be an example of the information processing system 200 described with reference to FIG. 2. As illustrated, the information processing system 1100 includes one or more processors 1110, a bus 1130, a communication interface 1140, and a memory 1120 for loading a computer program 1160 executed by a processor 1110. Meanwhile, only the components related to the present example are illustrated in FIG. 11. Accordingly, those of ordinary skill in the art to which the present disclosure pertains will be able to recognize that other general-purpose components may be further included in addition to the components illustrated in FIG. 11.

The processors 1110 control the overall operation of components of the information processing system (e.g., the information processing system 200). In present disclosure, the processor 1110 may be configured with a plurality of processors. The processor 1110 may include central processing unit (CPU), micro processor unit (MPU), micro controller unit (MCU), graphic processing unit (GPU), field programmable gate array (FPGA), at least two of any types of processors well known in the technical field of the present disclosure. In addition, the processors 1110 may perform an arithmetic operation on at least one application or program for executing the method according to various examples.

The memory 1120 may store various types of data, instructions, and/or information. The memory 1120 may load one or more computer programs 1160 in order to execute the method/operation according to various examples. The memory 1120 may be implemented as a volatile memory such as RAM, but the technical scope of the present disclosure is not limited thereto. For example, the memory 1120 may include a nonvolatile memory such as a read only memory (ROM), an erasable programmable ROM (EPROM), an electrically erasable programmable ROM (EEPROM), a flash memory, and the like, a hard disk, a detachable disk, or any type of computer-readable recording medium well known in the art to which the present disclosure pertains.

The bus 1130 may provide a communication function between components of the information processing system. The bus 1130 may be implemented as various types of buses such as an address bus, a data bus, a control bus, or the like.

The communication interface 1140 may support wired/wireless Internet communication of the information processing system. In addition, the communication interface 1140 may support various other communication methods in addition to the Internet communication. To this end, the communication interface 1140 may include a communication module well known in the technical field of the present disclosure.

The computer program 1160 may include one or more instructions that cause the processors 1110 to perform operations/methods in accordance with various examples. That is, the processors 1110 may perform operations/methods according to various examples by executing one or more instructions.

For example, the computer program 1160 may include one or more instructions for extracting patches from a medical image, generating a batch using a plurality of extracted patches, outputting or extracting an analysis result from the generated batch, generating/inferring medical information based on one or more extracted analysis results, and training a machine learning model to infer an analysis result and/or medical information from the medical image using the generated batch as training data, and the like. In this case, a system for processing medical images in parallel according to some examples may be implemented through the information processing system 1100.

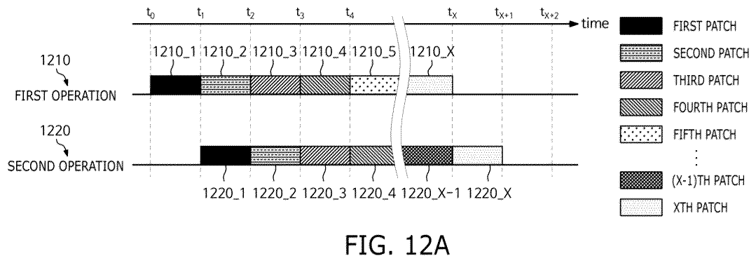

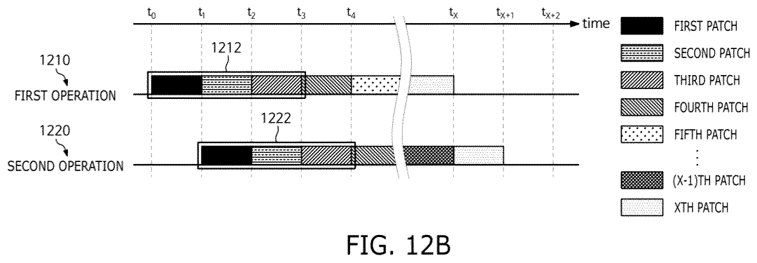

FIGS. 12A and 12B illustrate examples of parallel processing a medical image through asynchronous operations on a patch basis. Referring to FIGS. 12A and 12B, a first operation 1210 may be performed for each of X patches (where X is a natural number equal to or greater than 2) included in the medical image (e.g., digitally scanned pathology images). FIGS. 12A and 12B illustrate that six or more patches are included, but aspects are not limited thereto. For example, the medical image may include one to five patches.

The X patches may be extracted from the medical image by the first processor. In this case, the first processor may perform the first operation 1210 of providing the second processor with each of the extracted X patches. Alternatively, X patches may be previously extracted from the medical image and stored on a storage medium. In this case, the first processor may perform the first operation 1210 of extracting the X patches stored in the storage medium and providing them to the second processor. The X patches may be patches included in one medical image or patches included in a plurality of medical images. Additionally, the medical image may be stored in a memory accessible by the first processor (e.g., an internal memory of an information processing system, a memory included in an external device, etc.)

FIG. 12A illustrates a method for parallel processing a medical image through asynchronous operations on a patch basis. From t₀ to t₁, the first processor may perform a first operation 1210_1 for the first patch, by providing the second processor with the first patch included in one or more medical images. From t₁ to t₂, the first processor may perform a first operation 1210_2 for the second patch, by providing the second processor with the second patch included in one or more medical images. Further, from t₁ to t₂, the second processor may perform a second operation 1220_1 for the first patch, by outputting a first analysis result using the first patch received from the first processor. That is, from t₁ to t₂, while the first processor is performing the first operation 1210_2 for the second patch, the second processor may perform the second operation 1220_1 for the first patch. The second operation may include an operation of storing the output analysis result in the storage medium.

Accordingly, the time frame for the first operation 1210_2 for the second patch may overlap with the time frame for the second operation 1220_1 for the first patch performed by the second processor. That is, while the first processor is performing the first operation 1210_2 for the second patch, the second processor may process the second operation 1220_1 for the first patch in parallel. Specifically, while the first processor is performing the first operation 1210_2 for the second patch, at least a part of the second operation 1220_1 for the first patch of the second processor may be performed. Alternatively, while the second operation 1220_1 for the first patch of the second processor is being performed, the first processor may perform at least a part of the first operation 1210_2 for the second patch. FIGS. 12A and 12B illustrate, for convenience of explanation, all of the time frames for the first operation performed by the first processor and all of the time frames for the second operation performed by the second processor overlap with each other at a specific time, but aspects are not limited thereto, and at least a part of the time frame for the first operation performed by the first processor may overlap with at least a part of the time frame for the second operation performed by the second processor at a specific time.

Likewise, the time frame for the first operation 1210_3 for the third patch may overlap with the time frame for the second operation 1220_2 for the second patch performed by the second processor, and the time frame for the first operation 1210_4 for the fourth patch may overlap with the time frame for the second operation 1220_3 for the third patch performed by the second processor. That is, from tₓ₋₁ to tₓ, the first processor may perform a first operation 1210_X for the X patch, by providing the second processor with the X patch included in one or more medical images. Further, from tₓ₋₁ to tₓ, the second processor may perform the second operation 1220_X-1 for an (X-1)th patch, by outputting an (X-1)th patch analysis result by using the (X-1)th patch received from the first processor. That is, from tₓ₋₁ to tₓ, while the first processor is performing the first operation 1210_X for the Xth patch, the second processor may perform the second operation 1220_X-1 for the (X-1)th patch in parallel. Accordingly, the time frame for the first operation 1210_X of the Xth patch may overlap with the time frame for the second operation 1220_X-1 for the (X-1)th patch performed by the second processor.

A plurality of patches may be included in one batch. In addition, a plurality of patches included in one batch may be processed in parallel through asynchronous operations on a patch basis. For example, if the first operation 1210 for the first patch included in the first batch is completed, the second operation 1220 for the first patch included in the first batch may be performed before the first operation for the remaining patches included in the first batch is completed. Referring to FIG. 12B, the first to third patches may be included in first batches 1212 and 1222. Accordingly, from t₀ to t₁, if the first operation of providing the second processor with the first patches included in the first batches 1212 and 1222 is completed by the first processor, from t₁ to t₂, the second processor may perform the second operation for the first patches included in the first batches 1212 and 1222. At the same time, from t₁ to t₂, if the first operation of providing the second processor with the second patches included in the first batches 1212 and 1222 is completed by the first processor, from t₂ to t₃, the second processor may perform the second operation for the second patches included in the first batches 1212 and 1222. That is, before the first operation for the remaining patches included in the first batches 1212 and 1222 is completed, the second operation may be immediately performed for each patch for which the first operation was completed.

As described above, the second processor may perform the second operation for all patches included in the first batch without waiting for the first operation to be completed. That is, if the first operation for even one of the patches included in the first batch is completed, the second operation may be immediately performed for the corresponding patch. According to this configuration, the time required for processing the medical images can be shortened as much as possible. In addition, the waiting time of the processor, during which the processor does not perform operations, is shortened, and server operating costs can also be significantly reduced.

FIG. 12B illustrates that the first batches 1212 and 1222 include three patches, but the number of patches included in the batch is not limited thereto. That is, the number of patches included in the first batches 1212 and 1222 may be N (where N is a natural number). In addition, the number of patches included in each batch may be different. For example, the number of patches included in the first batch may be different from the number of patches included in the second batch.

For convenience of explanation, FIGS. 12A and 12B illustrate that the time required for the first operation and the time required for the second operation are the same, but aspects are not limited thereto. For example, the time required for the first operation for the first patch (e.g., first operation 1210_1, etc.) and the time required for the first operation for the second patch (e.g., first operation 1210_2, etc.) may be different from each other. In addition, the time required for the first operation for the first patch (e.g., first operation 1210_1, etc.) and the time required for the second operation for the first patch (for example, the second operation 1220_1, etc.) may be different from each other. In addition, the time required for the first operation for the second patch (e.g., first operation 1210_2, etc.) and the time required for the second operation for the first patch (for example, the second operation 1220_1, etc.) may be different from each other. If the time required for each operation for a patch is different from each other or if the time required for an operation for each patch is different from each other, the effect of reducing the time required for processing a medical image can be greater with the asynchronous operations on a patch basis.

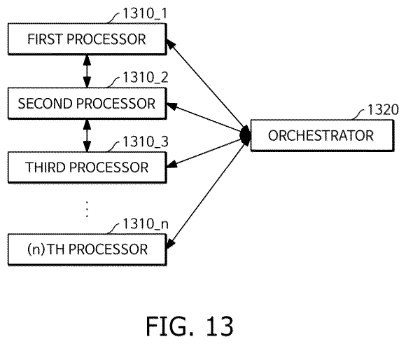

FIG. 13 is a structural diagram illustrating an example of an orchestrator 1320. As illustrated, a plurality of processors 1310_1 to 1310_n (where n is a natural number equal to or greater than 2) may be configured such that the processors are connected through the orchestrator 1320 and process commands of the orchestrator 1320. The orchestrator 1320 may check the status of the plurality of processors 1310_1 to 1310_n and determine an operation scale of processors to perform each operation. The orchestrator 1320 may refer to at least one arbitrary processor that determines the operation scale for the plurality of processors 1310_1 to 1310_n. FIG. 13 illustrates that the orchestrator 1320 is provided separately from the plurality of processors 1310_1 to 1310_n, but aspects are not limited thereto and the orchestrator 1320 may be included in at least one processor of the plurality of processors 1310_1 to 1310_n.

The orchestrator 1320 may calculate the throughput required for each of a plurality of operations for medical image processing for each of a plurality of batches which may be extracted from a medical image (e.g., a digitally scanned pathology image, etc.) or extracted from the medical image and stored. The required throughput (e.g., data size, data time, etc.) may be calculated based on the number of patches included in each batch (i.e., data size), the time required for processing each operation, etc. For example, the orchestrator 1320 may calculate the throughput required for the first operation for each of the first to Xth batches (where X is a natural number equal to or greater than 2). In addition, the orchestrator 1320 may calculate the throughput required for the second operation for each of the first to Xth batches. Likewise, the orchestrator 1320 may calculate the throughput required for the nth operation (where n is a natural number equal to or greater than 3) for each of the first to Xth batches.

The orchestrator 1320 may acquire the status of operations to be performed by each processor 1310_1 to 1310_n at a specific point in time. Specifically, the orchestrator 1320 may determine the status of the processors in active state and the processors in inactive state at the time each operation is requested. The “processor in active state” may refer to a processor that can immediately process the requested operation, and the “processor in inactive state” may refer to a processor that can process the requested operation after power is applied. For example, the processor in active state may refer to a processor that is powered on at the at the time of arrival of a request for operation but is not processing an operation and thus can immediately begin processing the operation. In addition, the processor in inactive state may refer to a processor that can process the requested operation after power is applied. That is, the orchestrator 1320 may acquire the status of the operations performed by each processor 1310_1 to 1310_n at the time of completion of each operation(or at a predetermined time interval, at the time when the state of at least one processor changes, at the time when a predetermined request is received) and check the available processors that can process the requested operation. For example, the orchestrator 1320 may determine the status of the processors (processors in active state) that are not processing operations as well as the processors (processors in inactive state) that are not powered.

The orchestrator 1320 may calculate the maximum throughput that the processor can process at the time of request for one or more operations. Specifically, the orchestrator 1320 may determine the status of the processors in active state at the time of request for one or more operations. Then, based on the amount of computation that the available processor can process, the orchestrator 1320 may calculate the maximum throughput that the processor in active state can process at the end of each operation.

The orchestrator 1320 may determine the operation scale of processors to process each operation. For example, the orchestrator 1320 may determine the operation scale of processors to process one or more requested operations based on the throughput required for the one or more requested operations and the maximum throughput that the processor in active state can process at the time of request for the one or more operations. Furthermore, the orchestrator 1320 may determine the operation scale of processors to process the one or more operations based on required throughput for the one or more requested operations and the maximum throughput that the processor in active state can process at the time of request for the one or more operations, as well as a target processing time by which each of the one or more operations must be completed.

For example, if the first operation for the first batch is completed and the second operation for the first batch is requested, the orchestrator 1320 may calculate the throughput required for the second operation for the first batch. In addition, the orchestrator 1320 may acquire the status of the operation to be processed by each of the plurality of processors 1310_1 to 1310_n at the time of request for the second operation for the first batch, and calculate the maximum throughput that the processor in active state can process. The orchestrator 1320 may determine the operation scale of processors to process the second operation for the first batch based on the throughput calculated for the second operation and the maximum throughput that can be processed by the plurality of processors 1310_1 to 1310_n at the time of request for the second operation. Likewise, if the second operation for the first batch is completed and the third operation is requested, the orchestrator 1320 may determine the operation scale of processors to process the third operation for the first batch. Likewise, the orchestrator 1320 may determine the operation scale of processors to process subsequent operations at the time of request for the operation for each batch included in the medical image. In this example, determining the operation scale of processors to process one operation at one point in time is described, but aspects are not limited thereto, and if a plurality of operations are requested at one point in time or within a predetermined range of time, it is possible to determine the operation scale of processors to process the plurality of operations.

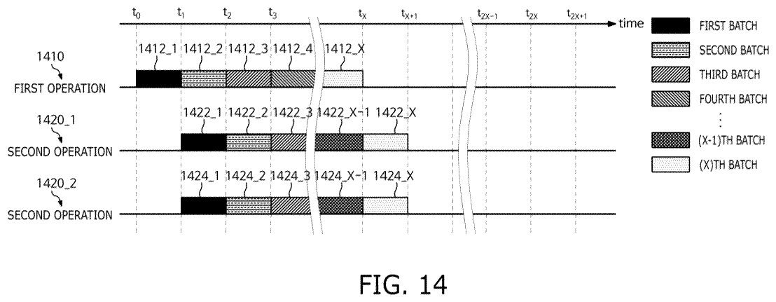

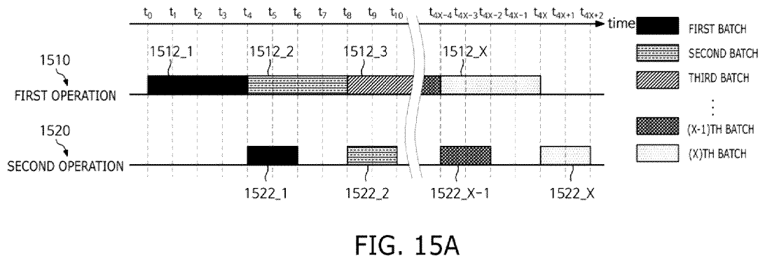

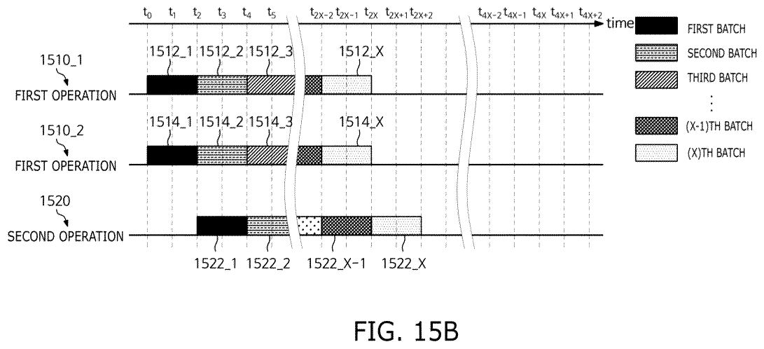

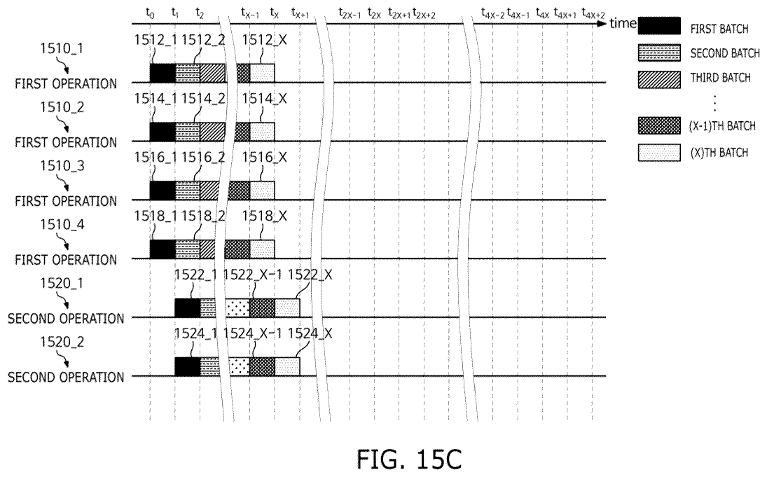

The orchestrator 1320 may allocate one or more processors to process each operation according to the determined operation scale of processors. For example, the orchestrator 1320 may allocate one or more processors to distribute and process the operation based on the throughput required for the operation. The scale of processors allocated by the orchestrator 1320 may be determined differently according to the service level agreement of the user who is using the method for parallel processing for medical images. A method of allocating one or more processors by the orchestrator 1320 will be described in detail below with reference to FIGS. 14 and 15A to 15C.

The orchestrator 1320 may determine activation and/or deactivation of each of a given plurality of processors according to the determined operation scale of processors. For example, if the maximum throughput of the processor in active state is smaller than the determined operation scale of processors, the orchestrator 1320 may determine re-activation of the processor in inactive state. In this case, the orchestrator 1320 may generate a command to power on the processor for which re-activation is determined. In this case, the orchestrator 1320 may directly provide the processor with the command to apply power, or provide a device that controls the processor with the command Conversely, if the maximum throughput that can be processed by the processor in active state is greater than the determined operation scale of processors, the orchestrator 1320 may determine deactivation of some of the processors in active state. In this case, the orchestrator 1320 may directly provide the processor with a command to terminate the processor determined to be deactivated, or provide a device that controls the processor with the command. According to this configuration, the orchestrator 1320 adjusts the operation scale of processors for each operation in real time, enabling more efficient server operation. In addition, by turning off the power of a server running a processor that is not processing an operation and turning the power back on at the time of request for the operation, the operating cost of the server can be significantly reduced.

FIG. 14 illustrates an example of parallel processing medical images using the orchestrator. According to FIG. 14, first and second operations may be performed for X (where X is a natural number equal to or greater than 2) batches included in a medical image (e.g., a digitally scanned pathology image). Each batch may include a plurality of patches. FIG. 14 illustrates that six or more batches are included, but aspects are not limited thereto. For example, the medical image may include one to five batches. In addition, FIG. 14 illustrates that the operation of processing the medical image by the first and second operations, but aspects are not limited thereto, and the operation of processing the medical image may be divided into three or more operations. In this case as well, the technique of allocating each of three or more operations to each of a plurality of processors by the orchestrator described in FIG. 14 may be applied.

Each of the first operation and the second operation may be performed by one or more different processors. For example, the first operation may be performed by one or more first processors (not illustrated), and the second operation may be performed by one or more second processors (not illustrated). Additionally, the first operation and the second operation may refer to different operations for the medical images. The orchestrator may determine the number of processors to perform the first operation and the number of processors to perform the second operation for each batch. In addition, the first operation may be allocated to one or more processors to perform the first operation and the second operation may be allocated to one or more processors to perform the second operation may be allocated based on the determined number of processors.

The amount of computation required for processing the first operation for one batch may be different from the amount of computation required for processing the second operation for one batch. Accordingly, the time required for the first operation for one batch and the time required for the second operation for one batch may be different from each other. The orchestrator may determine the number of processors to process the operations based on the time required for each operation for one batch. In addition, each operation may be allocated to one or more processors based on the determined number of processors.

FIG. 14 illustrates an example where the time required for a first operation 1410 for one batch is shorter than the time required for second operations 1420_1 and 1420_2 for one batch. In this case, the processor may determine the number of processors to perform the first operation 1410 to be smaller than the number of processors to perform the second operations 1420_1 and 1420_2.

The orchestrator may determine the number of processors to perform the first operation 1412_1 for the first batch at to, that is, at the time of request for the first operation 1412_1. According to the determined number of processors, from t₀ to t₁, the first processor may perform the first operation 1412_1 for the first batch. At time t₁, the orchestrator may determine the number of processors to perform a first operation 1412_2 for a second batch. According to the determined number of processors, from t₁ to t₂, the first processor (not illustrated) may perform the first operation 1412_2 for the second batch. In addition, at time t₁, the orchestrator may determine the number of processors to perform the second operations 1422_1 and 1424_1 for the first batch. According to the determined number of processors, from t₁ to t₂, the second processor (not illustrated) and the third processor (not illustrated) may perform the second operations 1422_1 and 1424_1 for the first batch in parallel, respectively. That is, from t₁ to t₂, while the first processor is performing the first operation 1412_2 for the second batch, the second processor and the third processor may perform the second operations 1422_1 and 1424_1 for the first batch, respectively. Accordingly, the time frame of the first operation 1412_2 for the second batch may overlap with the time frame of the second operations 1422_1 and 1424_1 for the first batch. That is, while the first processor is performing the first operation 1412_2 for the second batch, the second processor and the third processor process the second operations 1422_1 and 1424_1 for the first batch, respectively in parallel. FIG. 14 illustrates that all of the time frame of the first operation 1412_2 for the second batch overlap with all of the time frames of the second operations 1422_1 and 1424_1 for the first batch, but aspects are not limited thereto, and at least a part of the time frame of the first operation 1412_2 for the second batch may overlap with at least a part of the time frames of the second operations 1422_1 and 1424_1 for the first batch.

Likewise, from t₂ to t₃, while the first processor is performing a first operation 1412_3 for the third batch, the second processor and the third processor may perform the second operations 1422_2 and 1424_2 for the second batch, respectively. In addition, from t₃ to t₄, while the first processor is performing a first operation 1412_4 for the fourth batch, the second processor and the third processor may perform second operations 1422_3 and 1424_3 for the third batch, respectively. Under this operation, from tₓ₋₁ to tₓ, while the first processor is performing a first operation 1412_X for the Xth batch, the second processor and the third processor may respectively perform second operations 1422_X-1 and 1424_X-1 for the (X-1)th batch, respectively. In FIG. 14, from tₓ to tₓ₊₁, while the first processor performs no operation, from tₓ to tₓ₊₁, the second processor and the third processor may perform second operations 1422_X and 1424_X for the Xth batch.

The processors performing the first operation 1410 and the second operations 1420_1 and 1420_2 may be allocated differently according to each batch. For example, the first operation 1412_1 for the first batch may be performed by the first processor, the first operation 1412_2 for the second batch may be performed by the second processor, and the first operation 1412_3 for the third batch may be performed by the third processor. Alternatively, the first processor may again perform the first operation 1412_3 for the third batch. That is, the orchestrator may allocate the processors to perform subsequent operations based on the status of the processors (e.g., based on the status of processors in active state, based on processors in inactive state, etc.) at the time of request for each operation.

The orchestrator may turn off the power of the processor if the operation is completed. For example, at time tₓ when the first operation 1412_X for batch X (where X is a natural number) is finished and the first operation 1412_X+1 is requested, the orchestrator may turn off the power of the first processor that processed the Xth batch. In addition, at tₓ₊₁, that is, at a time point when the second operations 1422_X and 1424_X for the Xth batch are completed, the power of the second and third processors that respectively processed the second operations 1422_X and 1424_X may be turned off. Alternatively, the orchestrator may turn off the power of the second processor that processed the second operation 1422_X-1 at tX, that is, at the time point when the second operation 1422_X-1 for the (X-1)th batch is completed, and, from tₓ to tₓ₊₁, may control the first processor and the third processor to perform the second operations 1422_X and 1424_X for the Xth batch. Alternatively, the orchestrator may turn off the power of the third processor that processed the second operation 1424_X-1 for the (X-1)th batch at tₓ, that is, at the time point when the second operation 1424_X-1 for the (X-1)th batch is completed, and, from tₓ to tₓ₊₁, may control the first and second processors to perform the second operations 1422_X and 1424_X for the Xth batch, respectively. That is, the orchestrator may allocate the processors, of the processors in active state, to perform subsequent operations and turn off the power of the processor that completed the operation.

For convenience of description, FIG. 14 illustrates an example where one processor performs the first operation 1410 and two processors perform the second operation 1420_1 and 1420_2 which are distributed into two sub-operations, but aspects are not limited thereto, and the first operation may be performed by (n) processors (where n is a natural number equal to or greater than 2), and the second operation may be performed by (n+m) processors (where n and m are natural numbers). In this case, the first operation may be divided into (n) sub-operations and allocated to each of the (n) processors processing, and the second operation may be divided into (n+m) sub-operations and allocated to each of the (n+m) processors for processing. Alternatively, there may be (n) processors performing the first operation and (n) processors performing the second operation (where n is a natural number). That is, the number or scale of processors performing each operation is determined by the orchestrator based on the amount of computation required for each operation to process the batch and the maximum throughput of at least some of the processors.

According to this configuration, the orchestrator may allocate the operations such that one or more processors may process the operations in parallel based on the amount of computation required for each operation, so that the time required for processing the medical images can be shortened as much as possible. In addition, the orchestrator can determine the status of the operations to be processed by each processor in real time and adjust the operation scale of processors to process subsequent operations, thereby maximizing the efficiency of operation processing.

In addition, FIG. 14 illustrates that each of a plurality of processors is allocated based on the amount of computation required for each operation, the maximum throughput of available processors, etc. at the time of request for each operation (e.g., at t₀, t₁, t₂, t₃, . . . , tₓ, tₓ₊₁) by the orchestrator, but aspects are not limited thereto, and, if the orchestrator receives a request to process some or all of a plurality of batches extracted from medical images, the orchestrator may analyze the batches requested to be processed and determine the processing time and processors to be used for processing each of the batches requested to be processed.

FIGS. 15A to 15C illustrate an example of allocating processors based on the service level agreement of the user. At a specific point in time (e.g. at a time point of request for operation), the orchestrator may determine the operation scale of processors to perform each operation for the batch included in the medical image. At this time, the operation scale of processors may be determined based on the throughput required for each operation, the status of operations to be processed by a plurality of processors at the time of request for processing, and the target processing time. The “target processing time” as used herein may refer to a total time required for processing the medical image.

The target processing time may be determined differently according to the service level agreement of the user who is using the method for parallel processing a medical image. Accordingly, the processors allocated to perform each operation, that is, the operation scale of processors, may be determined differently according to the service level agreement of the user. For example, if a relatively short target processing time is set, a relatively large scale of processors may be determined for each operation. Accordingly, the orchestrator may increase the number of processors operating in parallel to shorten the total time required for processing medical images. Conversely, if a relatively long target time is set, a relatively small scale of processors may be determined for each operation. Accordingly, the orchestrator may decrease the number of processors operating in parallel, thereby reducing server operating costs per unit time, at the expense of increasing the total time required for processing medical images.

FIGS. 15A to 15C illustrate an example in which the number of processors is determined differently as the target processing time is set differently according to the service level agreement of the user. FIGS. 15A to 15C illustrate an example in which the time required for the first operation for one batch is longer than the time required for the second operation for one batch. While FIGS. 15A to 15C illustrate that the times required for the first operation for one batch are the same, aspects are not limited thereto. For example, the time required for the first operation for the first batch may be different from the time required for the first operation for the second batch. Likewise, while it is illustrated that the times required for the second operation for one batch are the same, aspects are not limited thereto. For example, the time required for the second operation for the first batch may be different from the time required for the second operation for the second batch.

It may be assumed herein that there are three service level agreements of the user, although aspects are not limited thereto. FIG. 15A illustrates an example in which the user corresponds the lowest service level agreement of the three service level agreements, in which case two processors are allocated to the first and second operations which are at least some of the operations for processing medical images so as to process the medical images in parallel. The orchestrator may determine the number of processors to perform a first operation 1512_1 for the first batch. According to the determined number of processors, from t₀ to t₄, the orchestrator may control the first processor (not illustrated) to perform the first operation 1512_1 for the first batch. The orchestrator may determine the number of processors to perform a first operation 1512_2 for the second batch. According to the determined number of processors, from t₄ to t₈, the first processor may perform the first operation 1512_1 for the second batch. Further, The orchestrator may determine the number of processors to perform a second operation 1522_1 for the first batch. According to the determined number of processors, from t₄ to t₆, the second processor (not illustrated) may perform the second operation 1522_1 for the first batch. That is, from t₄ to t₈, while the first processor is performing the first operation 1512_2 for the second batch, from t₄ to t₆, the second processor may perform the second operation 1522_1 for the first batch. Accordingly, at least a part of the time frame of the first operation 1512_2 for the second batch may overlap with the time frame of the second operation 1522_1 for the first batch. That is, while the first processor is performing the first operation 1512_2 for the second batch, the second processor may process the second operation 1522_1 for the first batch in parallel.

Likewise, from t₈ to t₁₂, while the first processor is performing the first operation 1512_2 for the second batch, from t₈ to t₁₀, the second processor may perform the second operation 1522_1 for the first batch. Likewise, from t_{4X-4} (where X is a natural number equal to or greater than 4) to t_{4X}, while the first processor is performing a first operation 1512_X for the Xth batch, from t_{4X-4} to t_{4X-2}, the second processor may perform a second operation 1522_X-1 for the (X-1)th batch. The first processor may not perform any operation from t_{4X} to t_{4X+2}. In addition, from t_{4X} to t_{4X+2}, the second processor may perform the second operation 1522_X for the Xth batch. As a result, the total time required for processing medical images in parallel using two processors may be the time from t₀ to t_{4X+2}.

FIG. 15B illustrates an example in which the user corresponds to a middle service level agreement of the three service level agreements, in which case three processors are allocated to process medical images in parallel. As illustrated, the total time required for the method for processing medical images in parallel using the three processors illustrated in FIG. 15B may be shorter than the total time required for the method for processing medical images in parallel using two processors illustrated in FIG. 15A.

Referring to FIG. 15B, the orchestrator may determine the number of processors to perform the first operations 1512_1 and 1514_1 for the first batch. According to the determined number of processors, from t₀ to t₂, the orchestrator may control the first and second processors to perform the first operations 1512_1 and 1514_1 for the first batch, respectively. The orchestrator may determine the number of processors to perform the first operations 1512_2 and 1514_2 for the second batch. According to the determined number of processors, from t₂ to t₄, the first and second processors may respectively perform the first operations 1512_2 and 1514_2 for the second batch. Further, the orchestrator may determine the number of processors to perform a second operation 1522_1 for the first batch. According to the determined number of processors, from t₂ to t₄, the third processor may perform the second operation 1522_1 for the first batch. That is, from t₂ to t₄, while the first and second processors each distribute and perform the first operation 1512_2 for the second batch, the third processor may perform the second operation 1522_1 for the first batch.

Likewise, from t₄ to t₆, while the first and second processors are performing the first operations 1512_3 and 1514_3 for the third batch, the third processor may perform the second operation 1522_2 for the second batch. Likewise, from t_{2X-2} (where X is a natural number equal to or greater than 4) to t_{2X}, while the first and second processors are performing the first operations 1512_X and 1514_X for the X-th batch, the third processor may perform the second operation 1522_X-1 for the (X-1)th batch. The first and second processors may not perform any operation from t_{2X} to t_{2X+2}. In addition, from t_{2X} to t_{2X+2}, the third processor may perform the second operation 1522_X for the Xth batch. As a result, the total time required for processing medical images in parallel using two processors may be the time from t₀ to t_{2X+2}. FIG. 15C illustrates an example in which the user corresponds to the highest service level agreement of the three service level agreements, in which case six processors are allocated to process medical images in parallel. As illustrated, the total time required for the method for processing medical images in parallel using six processors illustrated in FIG. 15C may be shorter than the total time required for the methods illustrated in FIGS. 15A and 15B. Referring to FIG. 15C, the orchestrator may determine the number of processors to perform the first operations 1512_1 to 1518_1 for the first batch. According to the determined number of processors, from t₀ to t₁, each of the first to fourth processors may perform the first operation 1512_1, 1514_1, 1516_1, and 1518_1 for the first batch. The orchestrator may determine the number of processors to perform the first operations 1512_2, 1514_2, 1516_2, and 1518_2 for the second batch. According to the determined number of processors, from t₁ to t₂, each of the first to fourth processors may respectively perform the first operations 1512_2 to 1518_2 for the second batch. In addition, the orchestrator may determine the number of processors to perform the second operations 1522_1 and 1524_1 for the first batch. According to the determined number of processors, from t₁ to t₂, each of the fifth processor and the sixth processor may distribute and perform the second operations 1522_1 and 1524_1 for the first batch. That is, from t₁ to t₂, while each of the first processor to the fourth processor distributes and performs the first operations 1512_2 to 1518_2 for the second batch, each of the fifth processor and the sixth processor may perform second operations 1522_1 and 1524_1 for the first batch.

Likewise, from tₓ₋₁ (where X is a natural number equal to or greater than 4) to tₓ, while each of the first to fourth processors is performing the first operations 1512_X and 1518_X for the Xth batch, each of the fifth processor and the sixth processor may perform the second operations 1522_X-1 and 1524_X-1 for the (X-1)th batch. In addition, from tₓ to tₓ₊₁, the first to fourth processors may not perform any operation and each of the fifth and sixth processors may perform the second operations 1522_X and 1524_X for the Xth batch. As a result, the total time required for processing medical images in parallel using six processors may be the time from t₀ to tₓ₊₁.

Referring to FIGS. 15A to 15C, by processing given operations in parallel, the number of the processors, from among the given processors, which are not performing operations, and the time the processors do not perform operations may be reduced or minimized. Moreover, the orchestrator may turn off the power of processors that are not performing operation. Under this operating environment, the time during which the processor does not perform operation during processing of medical images in parallel can be shortened or minimized. In addition, the total time required for processing medical images in parallel is shortened and the given processors can be operated efficiently.

With this configuration, the orchestrator may manage the operation scale of processors to process medical images differently for each user according to the service level agreements of the users. The users may select the service level agreements suitable for them, considering the time required for processing medical images, server operating costs, etc. The orchestrator may set different target processing times for processing of medical images in parallel according to the service level agreements of the user and provide a method for processing images in parallel suitable for the user's needs.

In addition, FIGS. 15A to 15C illustrate that each of a plurality of processors is allocated according to the service level agreement of the user based on the amount of computation required for each operation, the maximum throughput of available processors, etc. at the time of request for each operation (e.g., at t₀, t₁, t₂, t₃, . . . , tₓ, tₓ₊₁, . . . , t_{4X}, t_{4X+1}, t_{4X+2}) by the orchestrator, but aspects are not limited thereto, and, if the orchestrator receives a request to process some or all of a plurality of batches extracted from medical images, the orchestrator may analyze the batches requested to be processed and determine the processing time and processors to be used for processing each of the batches requested to be processed.

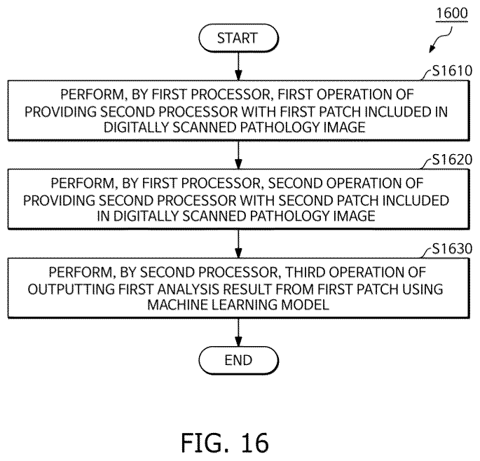

FIG. 16 is a flowchart illustrating another example of a method for processing medical images in parallel. The method 1600 may be performed by a plurality of processors (e.g., the plurality of processors 210_1 to 210_N in FIG. 2, etc.) The method 1600 may begin by a first processor performing a first operation of providing the second processor with a first patch included in a digitally scanned pathology image, at S1610. In this case, the performing the first operation may include extracting the first patch from the digitally scanned pathology image. Additionally or alternatively, the performing the first operation may include acquiring, as the first patch, one of a plurality of patches previously extracted from a digitally scanned pathology image and stored in a storage medium.

A second operation may be performed by the first processor, by providing the second processor with a second patch included in the digitally scanned pathology image, at S1620. The first and second patches may be included in one batch. In this case, the performing the second operation may include extracting the second patch from the digitally scanned pathology image. Additionally or alternatively, the performing the second operation may include acquiring, as the second patch, one of a plurality of patches previously extracted from a digitally scanned pathology image and stored in a storage medium. The first and second patches may be different from each other.

A third operation may be performed by the second processor, by outputting the first analysis result from the first patch using a machine learning model, at S1630. At this time, at least a part of the time frame for the second operation performed by the first processor may overlap with at least a part of the time frame for the third operation performed by the second processor. In addition, a fourth operation may be performed by the second processor, by outputting a second analysis result from the second patch using a machine learning model. Additionally, the medical information associated with the digitally scanned pathology image may be generated based on the first analysis result and the second analysis result.

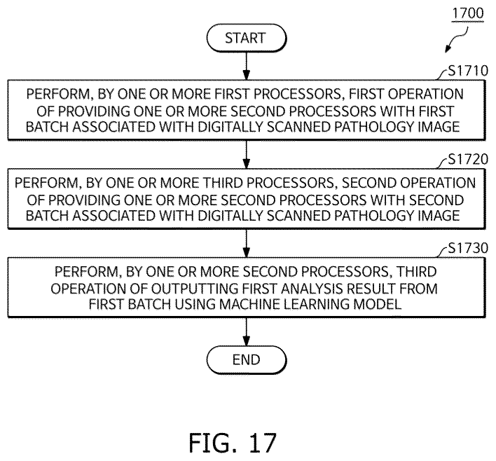

FIG. 17 is a flowchart illustrating an example of a method for parallel processing medical images using an orchestrator. The method 1700 may be performed by a plurality of processors (e.g., the plurality of processors 210_1 to 210_N in FIG. 2, etc.) The method 1700 may begin by one or more first processors performing a first operation of providing one or more second processors with a first batch associated with a digitally scanned pathology image, at S1710. The first batch may include a first set of patches.

The performing the first operation may include extracting a first set of patches from the digitally scanned pathology image and generating a first batch. Alternatively, the performing the first operation may include acquiring a first set of patches from a plurality of patches extracted from a digitally scanned pathology image and generating a first batch using the acquired first set of patches. The plurality of extracted patches may be stored in a storage medium in advance.

A second operation may be performed by one or more third processors, by providing the one or more second processors with a second batch associated with the digitally scanned pathology image, at S1720. The second batch may include a second set of patches. In addition, the first set of patches and the second set of patches may be different from each other. Additionally, at least a part of the one or more first processors may be the same as at least a part of the one or more third processors.

The performing the second operation may include extracting a second set of patches from the digitally scanned pathology image and generating a second batch. Alternatively, the performing the second operation may include acquiring a second set of patches from a plurality of patches extracted from a digitally scanned pathology image and generating a second batch using the acquired second set of patches. The plurality of extracted patches may be stored in a storage medium in advance.

A third operation may be performed by one or more second processors, by outputting the first analysis result from the first batch using a machine learning model, at S1730. At this time, at least a part of the time frame for the second operation performed by one or more third processors may overlap with at least a part of the time frame for the third operation performed by one or more second processors.

The method 1700 may further include calculating, by the orchestrator, the required throughput for the first operation, second operation, and third operation, and acquiring, from the given plurality of processors, the status of the operation to be processed by each of the plurality of processors at a specific point in time. The plurality of processors may include one or more processors in active state and one or more processors in inactive state.

The acquiring the status of the operation may include calculating a maximum throughput that can be processed by the one or more processors in active state at the time of request for processing of each of the first, second, and third operations. At this time, the “active state” may refer to a state in which each of the one or more processors can immediately process the requested operation. For example, the “processor in active state” may refer to a processor that is powered, and the “processor in inactive state” may refer to a processor that is not powered.

The method 1700 may include allocating, by the orchestrator, one or more processors of the plurality of processors to process each of the first operation, the second operation, and the third operation, based on the calculated throughput and the acquired status of the operations. The one or more processors allocated to process the first operation may be the one or more first processors, the one or more processors allocated to process the second operation may be the one or more third processors, and the one or more processors allocated to process the third operation may be the one or more second processors.

The allocating one or more processors may include determining deactivation of each of the one or more processors in active state or re-activation of each of the one or more processors in inactive state, based on the calculated throughput, the calculated maximum throughput, and the target processing time. At this time, the determining the deactivation of each of the one or more processors in active state or the re-activation of each of the one or more processors in inactive state may include, when re-activation of each of the one or more processors in inactive state is determined, applying power to each of the one or more processors for which the re-activation is determined, and when deactivation of each of the one or more processors in active state is determined, turning off each of the one or more processors for which the deactivation is determined. In addition, the target processing time may be determined differently according to the service level agreement of a user who is using the method for parallel processing the digitally scanned pathology image.

The above description of the present disclosure is provided to enable those skilled in the art to make or use the present disclosure. Various modifications of the present disclosure will be readily apparent to those skilled in the art, and the general principles defined herein may be applied to various modifications without departing from the spirit or scope of the present disclosure. Thus, the present disclosure is not intended to be limited to the examples described herein but is intended to be accorded the broadest scope consistent with the principles and novel features disclosed herein.

Although example implementations may refer to utilizing aspects of the presently disclosed subject matter in the context of one or more standalone computer systems, the subject matter is not so limited, and they may be implemented in conjunction with any computing environment, such as a network or distributed computing environment. Furthermore, aspects of the presently disclosed subject matter may be implemented in or across a plurality of processing chips or devices, and storage may be similarly influenced across a plurality of devices. Such devices may include PCs, network servers, and handheld devices. Although the present disclosure has been described in connection with examples herein, it should be understood that various modifications and changes can be made without departing from the scope of the present disclosure, which can be understood by those skilled in the art to which the present disclosure pertains. In addition, such modifications and changes should be considered within the scope of the claims appended herein.

1. A method for parallel processing a digitally scanned pathology image, the method being performed by a plurality of processors and comprising:
performing, by a first processor, a first operation of providing a second processor with a first patch included in the digitally scanned pathology image;
performing, by the first processor, a second operation of providing a second processor with a second patch included in the digitally scanned pathology image; and
performing, by the second processor, a third operation of outputting a first analysis result from the first patch using a machine learning model,
wherein at least a part of a time frame for the second operation performed by the first processor overlaps with at least a part of a time frame for the third operation performed by the second processor.

2. The method according to claim 1, further comprising performing, by the second processor, a fourth operation of outputting a second analysis result from the second patch using the machine learning model,
wherein medical information associated with the digitally scanned pathology image is generated based on the first analysis result and the second analysis result.

3. The method according to claim 1, wherein the first and second patches are included in one batch.

4. The method according to claim 1, wherein the performing the first operation includes extracting the first patch from the digitally scanned pathology image, the performing the second operation includes extracting the second patch from the digitally scanned pathology image, and
the first and second patches are different from each other.

5. The method according to claim 1, wherein the performing the first operation includes acquiring, as the first patch, one of a plurality of patches previously extracted from the digitally scanned pathology image and stored in a storage medium,
the performing the second operation includes acquiring, as the second patch, one of a plurality of patches previously extracted from the digitally scanned pathology image and stored in the storage medium, and
the first and second patches are different from each other.

6. A method for parallel processing a digitally scanned pathology image, the method being performed by a plurality of processors and comprising:
performing, by one or more first processors, a first operation of providing one or more second processors with a first batch associated with a digitally scanned pathology image, wherein the first batch includes a first set of patches;
performing, by one or more third processors, a second operation of providing the one or more second processors with a second batch associated with the digitally scanned pathology image, wherein the second batch includes a second set of patches; and
performing, by the one or more second processors, a third operation of outputting a first analysis result from the first batch using a machine learning model,
wherein at least a part of a time frame for the second operation performed by the one or more third processors overlaps with at least a part of a time frame for the third operation performed by the one or more second processors.

7. The method according to claim 6, further comprising:
calculating a throughput required for the first operation, the second operation, and the third operation;
acquiring, from a given plurality of processors, a status of operations to be processed by each of the plurality of processors at a specific point in time; and
allocating one or more processors of the plurality of processors to process each of the first operation, the second operation, and the third operation, based on the calculated throughput and the acquired status of the operations,
wherein the one or more processors allocated to process the first operation are the one or more first processors, the one or more processors allocated to process the second operation are the one or more third processors, and the one or more processors allocated to process the third operation are the one or more second processors.

8. The method according to claim 7, wherein the plurality of processors include one or more processors in active state and one or more processors in inactive state,
the acquiring the status of the operation includes calculating a maximum throughput that can be processed by the one or more processors in active state at the time of request for processing of each of the first operation, the second operation, and the third operation, and
the allocating the one or more processors includes determining deactivation of each of the one or more processors in active state or re-activation of each of the one or more processors in inactive state based on the calculated throughput, the calculated maximum throughput, and a target processing time.

9. The method according to claim 8, wherein the determining the deactivation of each of the one or more processors in active state or the re-activation of the one or more processors in inactive state includes:
if determining the re-activation of each of the one or more processors in inactive state, applying a power to each of the one or more processors for which re-activation is determined; and
if determining the deactivation of each of the one or more processors in active state, turning off the power of each of the one or more processors for which the deactivation is determined.

10. The method according to claim 8, wherein the target processing time is determined differently according to a service level agreement of a user who is using the method for parallel processing the digitally scanned pathology image.

11. The method according to claim 6, wherein the performing the first operation includes extracting the first set of patches from the digitally scanned pathology image to generate the first batch,
the performing the second operation includes extracting the second set of patches from the digitally scanned pathology image to generate the second batch, and
the first set of patches and the second set of patches are different from each other.

12. The method according to claim 6, wherein the performing the first operation includes:
acquiring the first set of patches from a plurality of patches extracted from the digitally scanned pathology image, wherein the extracted plurality of patches are previously stored in a storage medium; and
generating the first batch using the acquired first set of patches,
the performing the second operation includes:
acquiring the second set of patches from a plurality of patches extracted from the digitally scanned pathology image, wherein the extracted plurality of patches being previously stored in a storage medium; and
generating the second batch using the acquired second set of patches, and
the first set of patches and the second set of patches are different from each other.

13. The method according to claim 6, wherein, at least a part of the one or more first processors is the same as at least a part of the one or more third processors.

14. An information processing system, comprising:
a memory; and
one or more first processors, one or more second processors, and one or more third processors, which are connected to the memory and configured to execute at least one computer-readable program included in the memory,
wherein the at least one program includes instructions for:
performing, by the one or more first processors, a first operation of providing one or more second processors with a first batch associated with a digitally scanned pathology image, wherein the first batch includes a first set of patches;
performing, by the one or more third processors, a second operation of providing the one or more second processors with a second batch associated with the digitally scanned pathology image, wherein the second batch includes a second set of patches; and
performing, by the one or more second processors, a third operation of outputting a first analysis result from the first batch by using a machine learning model, and at least a part of a time frame for the second operation performed by the one or more third processors overlaps with at least a part of a time frame for the third operation performed by the one or more second processors.

15. The information processing system according to claim 14, wherein the at least one program further includes instructions for:
calculating a throughput required for the first operation, the second operation, and the third operation;
acquiring, from a given plurality of processors, a status of operations to be processed by each of the plurality of processors at a specific point in time; and
allocating one or more processors of the plurality of processors to process each of the first operation, the second operation, and the third operation, based on the calculated throughput and the acquired status of the operations, and
the one or more processors allocated to process the first operation are the one or more first processors, the one or more processors allocated to process the second operation are the one or more third processors, and the one or more processors allocated to process the third operation are the one or more second processors.

16. The information processing system according to claim 15, wherein the plurality of processors include one or more processors in active state and one or more processors in inactive state,
the acquiring the status of the operation includes calculating a maximum throughput that can be processed by the one or more processors in active state at the time of request for processing of each of the first operation, the second operation, and the third operation, and
the allocating the one or more processors includes determining deactivation of each of the one or more processors in active state or re-activation of each of the one or more processors in inactive state based on the calculated throughput, the calculated maximum throughput, and a target processing time.

17. The information processing system according to claim 16, wherein the determining the deactivation of each of the one or more processors in active state or the re-activation of each of the one or more processors in inactive state includes:
if determining the re-activation of each of the one or more processors in inactive state, applying a power to each of the one or more processors for which re-activation is determined; and
if determining the deactivation of each of the one or more processors in active state, turning off the power of each of the one or more processors for which the deactivation is determined.

18. The information processing system according to claim 16, wherein the target processing time is determined differently according to a service level agreement of a user who is using the method for parallel processing the digitally scanned pathology image.

19. The information processing system according to claim 14, wherein the performing the first operation includes extracting the first set of patches from the digitally scanned pathology image to generate the first batch,
the performing the second operation includes extracting the second set of patches from the digitally scanned pathology image to generate the second batch, and
the first set of patches and the second set of patches are different from each other.

20. The information processing system according to claim 14, wherein the performing the first operation includes:
acquiring the first set of patches from a plurality of patches extracted from the digitally scanned pathology image, wherein the extracted plurality of patches are previously stored in a storage medium; and
generating the first batch using the acquired first set of patches,
the performing the second operation includes:
acquiring the second set of patches from a plurality of patches extracted from the digitally scanned pathology image, wherein the extracted plurality of patches being previously stored in a storage medium; and
generating the second batch using the acquired second set of patches, and
the first set of patches and the second set of patches are different from each other.
